SEQUENCE LISTING (22 sequences)

SEQ ID NO: 1 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVYVSS

SEQ ID NO: 2 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 3 - protein, 5 aa, synthetic construct
SYGMH

SEQ ID NO: 4 - protein, 16 aa, synthetic construct
VIWEDGMKYYTDSVKG

SEQ ID NO: 5 - protein, 15 aa, synthetic construct
DRGIGARRGPYYMDV

SEQ ID NO: 6 - protein, 11 aa, synthetic construct
RASQSISSYLN

SEQ ID NO: 7 - protein, 7 aa, synthetic construct
AASSLQS

SEQ ID NO: 8 - protein, 9 aa, synthetic construct
QQSYSTPLI

SEQ ID NO: 9 - protein, 454 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 453 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGRYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
DIQMTWSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
SFGMH

SEQ ID NO: 14 - protein, 17 aa, synthetic construct
YISSGSSTIYYGDTVKG

SEQ ID NO: 15 - protein, 15 aa, synthetic construct
EGGYYYGRSYYTMDY

SEQ ID NO: 16 - protein, 16 aa, synthetic construct
RSSQSIVHSNGNTYLE

SEQ ID NO: 17 - protein, 7 aa, synthetic construct
KVSNRFS

SEQ ID NO: 18 - protein, 9 aa, synthetic construct
FQGSHVPPT

SEQ ID NO: 19 - protein, 112 aa, synthetic construct
DVVMTQSPLSLPXTPGXPASXSCRSSQSIVHSNGNTYLEWYLQKPGQSPKLLIYKVSNRFSGVPDRFSGSGSGTDFTLXIXXVXAEDVGIYYCFQGSHVPPTFGPGTKLEIK

SEQ ID NO: 20 - protein, 124 aa, synthetic construct
EVQLVESGGGLVQPGGSLRLSCSASGFTFSSFGMHWXXQXPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 21 - protein, 112 aa, synthetic construct
DVVMTQSPLSLPATPGDPASISCRSSQSIVHSNGNTYLEWYLQKPGQSPKLLIYKVSNRFSGVPDRFSGSGSGTDFTLTISRVDAEDVGIYYCFQGSHVPPTFGPGTKLEIK

SEQ ID NO: 22 - protein, 124 aa, synthetic construct
EVQLVESGGGLVQPGGSLRLSCSASGFTFSSFGMHWVRQTPGKGLEWVAYISSGSSTIYYGDTVKGRFTISRDNAKNSLFLQMSSLRAEDTAVYYCAREGGYYYGRSYYTMDYWGQGTTVTVSS

(19) United States
(12) Patent Application Publication
(10) Pub. No.: US 2026/0355128 A1
(43) Pub. Date: Nov. 12, 2026

(54) SCYLLO-INOSITOL IN COMBINATION WITH IMMUNOTHERAPEUTICS FOR THE TREATMENT OF ALZHEIMER'S DISEASE

(71) Applicant: EIRGEN PHARMA, LTD., Waterford (IE)

(72) Inventors: Phillip FROST, Miami Beach, FL (US); Antonio CRUZ, Miami, FL (US); John K. CHOE, Mississauga, Ontario (CA)

(21) Appl. No.: 19/109,143

(22) PCT Filed: Sep. 1, 2023
(86) PCT No.: PCT/US2023/031915

§ 371 Date: Mar. 6, 2025

Related U.S. Application Data
(60) Provisional application No. 63/441,732, filed on Jan. 27, 2023.
(60) Provisional application No. 63/453,583, filed on Mar. 21, 2023.

(51) Int. Cl. A61K 31/047; A61K 39/00; A61K 39/395; A61P 25/28; C07K 16/18

(52) U.S. Cl. CPC A61K 31/047; A61K 39/3955; A61P 25/28; C07K 16/18; A61K 2039/505

(57) ABSTRACT

The disclosure relates to a combination of active ingredients/adjuvants for the treatment of neurological disorders and diseases such as Alzheimer's disease and mild cognitive impairment (MCI) and memory and cognitive disorders and conditions. In particular, combinations of scyllo-inositol and treatments for Alzheimer's disease such as lecanemab are disclosed as useful.

### CROSS REFERENCE TO RELATED APPLICATIONS

This PCT application claims the benefit of U.S. Provisional Application No. 63/404,537, filed Sep. 7, 2022, U.S. Provisional Application No. 63/441,732, filed Jan. 27, 2023, and U.S. Provisional Application No. 63/453,583, filed Mar. 21, 2023, which all are herein incorporated by reference in their entireties.

### BACKGROUND OF THE INVENTION

Alzheimer's disease (AD) is a neurodegenerative disease or disorder that progresses over time to cause cognitive impairment and multiple symptoms and disabilities that impact the daily lives of subjects afflicted with this disease. The numbers of people living with this disease at its various stages is staggering and is estimated to increase to more than 115 million people worldwide by the year 2050. Effective treatments for the progression and/or treatment of Alzheimer's disease remain elusive despite years of effort and billions of dollars spent on drug development research. Before this year, only three drugs have been approved in the United States to treat this disease. These include donepezil, rivastigmine and galantamine-none of which are effective at stopping progression of the disease.

The FDA has, however, in a new development but with some reservations, recently approved the monoclonal antibodies drug aducanumab (BIIB037) and lecanemab for the treatment of mild cognitive impairment (MCI) and early Alzheimer's disease. Aducanumab and lecanemab are a recombinant, fully human, anti-amyloid-beta monoclonal antibodies. See U.S. Pat. No. 10,842,871 which is incorporated by reference herein. See also Sevigny J., et al. Nature, 537, 50-56 (2016). In the course of monoclonal antibody development in general for the treatment of AD, the FDA focused its attention on MRI abnormalities which were potentially associated with these treatments for Alzheimer's disease. MRI signal changes were thought to reflect vasogenic edema (VE) and possible microhemorrhages (mH). These MRI signal changes were observed in several clinical trials including Salloway 2009, Sperling 2009 and Black 2010).

LEQEMBI (lecanemab) (BAN2401) is indicated for intraveneous use as an amyloid beta-directed antibody for the treatment of Alzheimer's disease. Treatment with this drug should be initiated in patients having mild cognitive impairment or mild dementia stage of disease. The warnings and precautions section of the highlights of prescribing information also refers to amyloid related imaging abnormalities (ARIA) and enhanced clinical vigilance for ARIA is recommended during the first 14 weeks of treatment with LEQEMBI. Risk of ARIA, including symptomatic ARIA, was described as being increased in apolipoprotein E ε4 homozygotes compared to heterozygotes and noncarriers. Lecanemab-irmb is a recombinant humanized immunoglobulin gamma 1 (IgG1) monoclonal antibody directed against aggregated soluble and insoluble forms of amyloid beta. It is expressed in a CHO cell line and has an approximate molecular weight of 150 kDa. It is available commercially in single-dose vials at concentrations of 500 mg/5 mL (100 mgs/mL) or 200 mg/2 mL (100 mg/mL). The solution contains histidine hydrochloride monohydrate, polysorbate, histidine, arginine hydrochloride and water at a pH of 5.0.

The working group working with the FDA referred to a spectrum of image abnormalities that occur in the disease progression itself and in treatment thereof as Amyloid Related Imaging Abnormalities (ARIA). The terminology has progressed to refer to ARIA-E as those MRI signal alterations thought to represent VE and related extravasated fluid phenomena while ARIA-H means those signal alterations associated with mH and hemosiderosis. This focus on the underlying pathology seen in the MRI images led to the development of aducanumab and lecanemab as drugs which reduce the incidence of ARIA in susceptible Alzheimer's disease patients. In addition, the drugs aducanumab and lecanemab are approved for the treatment of mild Alzheimer's disease and are administered to a patient in increasing amounts over time. The preferred and approved drug of choice to treat Alzheimer's disease is now lecanemab. Clinical trials for lecanemab showed the monoclonal antibody targets soluble aggregated amyloid beta, with demonstrated activity across oligomers, protofibrils, and insoluble fibrils. Swanson, et al., Alzheimer's & Therapy (2021) 13:80.

The phase 2 trial on lecanemab aimed to establish the smallest dose that achieves ≥90% of the maximum treatment effect. The primary endpoint in the trial was described as a Bayesian analysis of 12-month clinical changes on the Alzheimer's Disease Composite Score (ADCOMS) for the 90% dose (ED90). The endpoint required an 80% probability of ≥25% reduction in decline versus placebo in the subjects. Secondary endpoints included 18-month Bayesian and frequentist analyses of brain amyloid reduction using positron emission tomography; clinical decline on ADCOMS Clinical Dementia Rating-Sum-of-Boxes (CDR-SB), and Alzheimer's Disease Assessment Scale-Cognitive Subscale (ADAS-Cog14); changes in CSF core biomarkers and total hippocampal volume (HV) using volumetric magnetic resonance imaging. The trials found in addition to data demonstrating a 64% probability to be better than placebo by 25% on ADCOMS at the 12-month date and some positive secondary outcomes at 18 months, lecanemab was well tolerated with only a 9.9% incidence of ARIA-edema/effusion at 10 mg/kg biweekly administration.

Doses of aducanumab, the other approved monoclonal antibody, are given in multiple doses of at least 1 mg/kg to said patients over periodic intervals. The increase in dosing amount may rise to 3 mg/kg and then 6 mg/kg and then 10 mgs/kg over the course of such periodic treatment. The dosing protocol for the administration of aducanumab or lecanemab may be based on ApoE4 status of the patient and each of the dosing intervals may be about 4 weeks apart. In what is described as a typical treatment method for aducanumab, 1-5 doses of 1 mg/kg are first administered at periodic intervals to a patient; this is then followed by the administration of 1-5 doses of 3 mg/kg at periodic intervals to such patient; and then followed by the administration of 1-5 doses of 6 mg/kg at such periodic intervals. Treatment with aducanumab or lecanemab reduces cerebral amyloid burden and also reduces the susceptibility of the patient to ARIA. Methods to determine the effectiveness of the drug include positron emission tomography (PET) composite standardized uptake ratio values (SUVR) by time point determined by PET scans in addition to MRI. Clinical Dementia Rating Sums of Boxes (CDR-SB) and Mini Mental State Examinations (MMSE) were used to assess the patient's progress on aducanumab. The clinical data in the studies on aducanumab and lecanemab showed amyloid plague reduction after drug treatment.

For LEQEMBI (lecanemab), once the presence of amyloid beta is confirmed, the recommend dosage is 10 mg/kg that must be diluted and then administered as an intravenous infusion over approximately one hour, once every two weeks. MRIs are required to assess ARIA prior to initiating treatment and prior to the 5^{th}, 7^{th} and 14^{th} infusions. Monoclonal antibodies directed against aggregated forms of beta amyloid, including LEQEMBI, are known to cause ARIA, characterized as ARIA with edema (ARIA-E) which is detectable on MRI scans as brain edema or sulcal effusions, as stated on the prescribing information. Reduction or elimination of clusters or aggregated forms of beta amyloid would reduce ARIA due to monoclonal antibody treatment.

Donanemab is another monoclonal antibody advancing in clinical trials. This drug specifically targets a specific modified form of amyloid β, N-terminally truncated pyroglutamate modified amyloid β. Phase 2 clinical trials showed a significant reduction in Alzheimer's disease-associated cerebral amyloid-plaque levels as measured by positron emission tomography (PET) relative to the placebo treated subjects. See Lancet, Comment Vol 2 (7), E395-E396, July 2021. See also New England Journal of Medicine 2021; 384:1691-1704. These clinical trials also reported that ARIA-E had a significantly higher rate of occurrence among subjects in the drug treated group (26.7%) than those subjects in the placebo group (0-8%). There is thus a need in this particular antibody treatment regimen for a companion drug such as scyllo-inositol to reduce amyloid plaque burden and potentially reduce ARIA related events. Other known monoclonal antibodies for the treatment of Alzheimer's disease are inclusive of bapineuzumab, gantenerumab, GSK933776, solanezumab, crenezumab and ponezumab, anyone of which can be combined with scyllo-inositol as described herein.

It is believed that the docking of Aβ fibrils to neuronal and glial cell membranes may be an early and intervenable step during the progression of Alzheimer's disease. It has also been postulated that glycolipids such as gangliosides may result in the stabilization and prevention of Ab fibril formation while phosphatidyl inositols may result in the acceleration of fibril formation. Scyllo-inositol (ELND005) has been disclosed as useful in the treatment or prevention of a condition of the central or peripheral nervous system including Alzheimer's disease. See U.S. Pat. No. 7,521,481 which is hereby incorporated by reference.

While there has been general disclosure in patent publications of some combination treatments of scyllo-inositol and other neurological focused drugs or therapies, there has been no disclosure of a combination of monoclonal antibodies selected from aducanumab or lecanemab and scyllo-inositol nor have there been any disclosures of the use of scyllo-inositol to reduce ARIA events when combined with or used as a pre-treatment for patients that are taking monoclonal antibodies to treat Alzheimer's disease or related cognitive disorders. Six clinical trials are shown for scyllo-inositol in ClinicalTrials.gov. Completed studies in Alzheimer's disease include the study entitled ELND005 in Patients with Mild to Moderate Alzheimer's disease and Long-term Follow-up Study in Subjects with Alzheimer's disease. The efficacy and safety of ELND005 as a treatment for agitation and aggression in Alzheimer's disease was also completed. A 36-week Safety Extension Study of ELND005 as a Treatment for Agitation and Aggression in Alzheimer's disease was terminated. The clinical trials included patients that received 250 mgs/BID of scyllo-inositol versus placebo. The primary outcomes in the Alzheimer's study was the change from baseline to week 78 in Cooperative Study-Activities of Daily Living (ADCS-ADL) Scores. The ADCS-ADL test is a 23-item scale that measures a subject's functional abilities as assessed by the subject's caregiver. The scale ranges from 0-78 with the lower scores suggesting functional impairment. Secondary outcomes included change in Alzheimer's disease Assessment Scale-Cognitive Subscale (ADAS-Cog) Score from baseline to week 78. This test measures cognitive ability and is composed of 12 items with scores ranging from 0 to 75. Higher scores suggest greater cognitive impairment. To date, following the results of such studies, scyllo-inositol has not advanced in a new drug application to treat Alzheimer's disease. While the results were not positive at the doses studied in the broad group of subjects studied, the present inventors have discovered a dosing regimen in combination with the monoclonal antibodies aducanumab or lecanemab that surprisingly and unexpectedly improves the clinical endpoints for these monoclonal antibodies in the treatment of Mild Cognitive Impairment (MCI) and mild to moderate Alzheimer's disease. The improvement primarily relates to the reduction of ARIA related events in subjects taking such monoclonal antibodies.

The rationale for such improvement may lie in the fact that scyllo-inositol is an oral drug that crosses the blood brain barrier achieving low mM levels and that the drug breaks down amyloid beta (Aβ) fibrils at low (0.1-5) nM levels. Scyllo-inositol also prevents binding of soluble Aβ and reduces neuronal toxicity. This drug has also been shown to improve memory/cognition in AD animal models and, in combination with aducanumab or lecanemab or other monoclonal antibodies used for the treatment of Alzheimer's, improves the side effect profile of such monoclonals such as aducanumab or lecanemab and permits the use of higher doses of monoclonal antibodies to more effectively treat MCI and Alzheimer's disease.

The use of scyllo-inositol in combination with monoclonal antibodies may also be useful to treat aging patients with memory loss due to increased accumulation of amyloid beta in the brain and MCI patients predisposed to develop mild to moderate Alzheimer's disease. These drug combinations will reduce amyloid accumulation in the brain, as well as reduce the amyloid inhibition of neuronal function. Pre-treatment with scyllo-inositol in those subjects prescribed to receive treatment with aducanumab or lecanemab will reduce levels of aggregated amyloid plaque and reduce monoclonal antibody associated ARIA while permitting higher doses of the monoclonal antibodies to further enhance Alzheimer's treatment.

### SUMMARY OF THE INVENTION

The invention comprises a method of treating MCI and Alzheimer's disease with a combination of scyllo-inositol and a monoclonal antibody. The preferred monoclonal antibody is lecanemab but others can include aducanamab, donanemab or any monoclonal antibody developed to treat Alzheimer's disease and which has ARIA related events associated with treatment using the monoclonal antibody. The combination products, in particular the combination products comprising scyllo-inositol and aducanumab or lecanemab, enhances aducanumnab's or lecanemab's efficacy in the treatment of MCI and mild AD patients. Scyllo-inositol also allows for a reduction in aducanumab's or lecanemab's dosing for the treatment of MCI and Mild AD while maintaining similar efficacy as a dose given without scyllo-inositol. Furthermore, scyllo-inositol reduces the prevalence of ARIA associated aducanumab or lecanemab treatment at the higher doses. Thus, the invention comprises a method for treating Alzheimer's disease in a human patient, comprising administering a pharmaceutically effective amount of a recombinant, fully human, anti-amyloid beta monoclonal antibody and a pharmaceutically effective amount of scyllo-inositol.

The invention also comprises a method of treating Alzheimer's disease or MCI with a combination of said recombinant monoclonal antibody selected from aducanumab or lecanemab (BAN2401) and scyllo-inositol (250 mgs once daily or BID or 500 mgs QD). The combination will reduce amyloid burden in the brain, reduce long-term decline in memory and cognitive function associated with aging; will improve membrane fluidity and increase neuronal function and improve both short and long-term memory and cognition.

In one embodiment, aducanumab comprises a heavy chain variable region (VH) with a light chain variable region (VL) wherein the V H comprises a complementarity determining region1 (VHCDR1) with the amino acid sequence set forth in SEQ ID NO: 3, a VHCDR2 with the amino acid sequence set forth in SEQ ID NO: 4, and a VHCDR3 with the amino acid sequence set forth in SEQ ID NO: 5, and wherein the VL comprises a VLCDR1 with the amino acid sequence set forth in SEQ ID NO: 6, a VLCDR2 with the amino acid sequence set forth in SEQ ID NO: 7, and a VLCDR3 with the amino acid sequence set forth in SEQ ID NO: 8.

In another embodiment, lecanemab comprises sequences selected from the group consisting of, subunit 1 (SEQ ID NO: 9); subunit 2 (SEQ ID NO: 9); subunit 3 (SEQ ID NO: 10); subunit 4 (SEQ ID NO: 10).

In an embodiment, scyllo-inositol optionally with an immunotherapeutic, is administered orally at a dosing range of between 100 to 250 mgs once daily or BID. In another embodiment, scyllo-inositol is administered as a dose of 500 mg dose once a day.

In an embodiment, the invention comprises a method of reducing brain amyloid beta plaque in Alzheimer's patients comprising the administration of an effective amount of aducanumab or lecanemab (lecanemab-irmb) in combination with an effective amount of scyllo-inositol.

In a further embodiment, the invention comprises a method of treating an Alzheimer's patient having the confirmed presence of amyloid pathology and mild cognitive impairment or mild dementia stage of disease consistent with Stage 3 or Stage 4 Alzheimer's disease comprising the administration of between about 1 mg/kg to about 10 mg/kg of aducanumab as an IV infusion over an hour period every four weeks and at least 21 days apart and comprising the administration of an effective amount of scyllo-inositol.

In a further embodiment, the invention comprises a method of treating an Alzheimer's patient having the confirmed presence of amyloid pathology and mild cognitive impairment or mild dementia stage of disease consistent with Stage 3 or Stage 4 Alzheimer's disease comprising the administration of between about 1 mg/kg to about 10 mg/kg of lecanemab as an IV infusion over an hour period once every two weeks and further comprising the administration of an effective amount of scyllo-inositol.

In another embodiment, the invention comprises a method according to the above embodiments wherein scyllo-inositol is administered once daily or BID at a strength of about 125 to 250 mgs in an oral dosage form.

In an embodiment, upon confirmation of the presence of amyloid beta pathology in a subject, the invention comprises (i) pre-treating the subject with a pharmaceutically effective amount of scyllo-inositol (ii) obtaining a brain MRI in the subject to evaluate for pre-existing Amyloid Related Imaging Abnormalities (ARIA) within one year of initiating treatment with a monoclonal antibody selected from lecanemab and (ii) administering a dosage of about 10 mg/kg of lecanemab in a diluted formulation and administering said diluted formulation of lecanemab as an intravenous infusion over approximately one hour, once every two weeks.

In an embodiment comprising subsequent infusions of lecanemab, the invention comprises continuing treatment with scyllo-inositol on a daily basis from the initiation of pre-treatment in a dosage amount of about 250-500 mgs BID or QD and obtaining MRIs prior to the 5^{th}, 7^{th} and 14^{th} infusions of lecanemab.

In a further embodiment, the lecanemab diluted formulation comprises lecanemab diluted in 250 mL of about 0.9% sodium chloride injection (USP) and administered by infusion via a terminal low-protein binding 0.2 micron in-line filter.

In another embodiment, the invention comprises a method wherein scyllo-inositol enhances the efficacy of aducanumab or lecanemab by permitting an increase in the dosing amount of aducanumab or lecanemab from about 10 mgs/kg to about 12-15 mgs/kg per infusion.

The invention further comprises a method of enhancing the cognition of a subject having MCI or mild Alzheimer's disease and in need of treatment thereof by (1) pretreating said subject with between 125-250 mgs once daily or BID with scyllo-inositol and (2) co-administering a pharmaceutically effective amount of scyllo-inositol in combination with aducanumab or lecanemab to said subject to enhance cognition in said subject.

The invention comprises a method of treating a patient in need of treatment thereof which comprises treating said patient with a combination of scyllo-inositol and aducanumab or lecanemab wherein following such treatment there is a reduction of ARIA aducanumab or lecanemab associated events for the combination treatment versus treatment with aducanumab or lecanemab alone at the same infusion doses.

The invention comprises a use of scyllo-inositol as an adjuvant for modifying the amount of a monoclonal antibody necessary to treat an Alzheimer's patient in need of treatment thereof.

In a preferred embodiment, the monoclonal antibody is selected from lecanemab.

In another preferred embodiment, the combination comprises scyllo-inositol and a pharmaceutically effective amount of lecanemab.

The invention also comprises a method of reducing ARIA in patients on monoclonal antibody treatment comprising administering a pharmaceutically effective amount of scyllo-inositol to the patient in need of treatment thereof wherein said reduction is in comparison to a patient on such monoclonal antibody treatment but without scyllo-inositol.

In an embodiment, the invention further comprises a method of decreasing amyloid beta burden in the brain of a patient who has mild Alzheimer's disease and is being treated with a monoclonal antibody selected from aducanumab or lecanemab comprising administering a pharmaceutically effective amount of scyllo-inositol to said patient.

In an embodiment, the invention comprises a method of improving memory, cognition and/or brain function in of an Alzheimer's patient in need of treatment thereof wherein the patient is being treated with a monoclonal antibody comprising co-administration of a pharmaceutically effective amount of scyllo-inositol wherein such co-administration results in improved memory, cognition and/or brain function relative to a patient being treated with the monoclonal antibody alone.

The invention further comprises a method of improving positive biomarkers in the CSF of an Alzheimer's patent being treated with a monoclonal antibody comprising the co-administration of a pharmaceutically effective amount of scyllo-inositol wherein said improvement is relative to a control patient being treated with the monoclonal antibody alone.

The invention also comprises a method according to any one of the embodiments above wherein the patient is pre-treated with scyllo-inositol before undergoing monoclonal antibody therapy at a daily dose BID of 125-250 mgs.

The invention comprises a method wherein the pre-treatment period is up to two weeks. In a preferred embodiment, such a combination would be sold as a packet having 250 mg BID or 500 mgs scyllo-inositol QD, and 10 mg/kg per infusion of a monoclonal antibody selected from aducanamab or lecanemab. The scyllo-inositol in such a combination is preferably in a dosage amount of 125-250 mgs BID but may also be provided QD in a dosage amount of 250 mgs to 500 mgs. Capsule sizes and types (hard versus soft) may also vary depending upon the amount of scyllo-inositol and the inactive ingredients.

### BRIEF DESCRIPTION OF THE DRAWINGS

FIGS. 1A-F shows the effect of 250 mgs scyllo-Inositol Treatment BID in Mild/Moderate AD Patients (MMSE 16-30) in the primary endpoints, NTB, ADCS-ADL, and CDR-SB

FIG. 2 demonstrates the effect of scyllo-inositol treatment of early mild AD patients (MMSE 23-26) for 78 weeks in a pre-specified full and per protocol populations.

FIGS. 3A-I shows NTB sub-item change from baseline to mild AD (PPS) in nine different sub-items.

FIG. 4 shows ADCS-ADL change from baseline for scyllo-inositol and placebo treated early-mild AD patients (MMSE 23-26) for 78 weeks.

FIG. 5: shows CDR-SB change from baseline for scyllo-inositol and placebo treated early mild AD patients (MMSE 23-26) for 78 weeks.

FIGS. 6A-F: shows the compares the effect of scyllo-inositol and placebo treatment on the CDR-SB sub-items change from baseline in early mild AD patients in the per protocol population (PPS).

FIGS. 7A-D shows the observed changes from baseline in NTB scores with scyllo-inositol treatment of mild AD patients with different MMSE scores ranging from 20-26, 21-26, 22-26 and 23-26 respectively.

FIGS. 8A-D shows Bootstrap simulated data for the change in NTB scores from baseline with scyllo-inositol treatment of different mild AD patient groups with MMSE scores ranging from 20-26, 21-26, 22-26 and 23-26 respectively.

FIGS. 9A-D shows the observed data demonstrating the changes in CDR-SB scores from baseline with scyllo-inositol treatment in different mild AD groups with an MMSE score of 20-26, 21-26, 22-26 and 23-26 respectively.

FIGS. 10A-D shows bootstrap simulated data demonstrating the changes in CDR-SB scores from baseline with scyllo-inositol treatment in different mild AD groups with an MMSE score range of 20-26, 21-26, 22-26 and 23-26 respectively.

FIGS. 11A-D shows a comparison of Observed and Bootstrap Simulated data for changes in NTB and CDR-SB scores from baseline for mild AD patients treated with scyllo-inositol having MMSE scores of 22-26.

### DETAILED DESCRIPTION

### Glossary

Numerical ranges recited herein by endpoints include all numbers and fractions subsumed within that range. (e.g. 1-5 includes 1, 1.5, 2, 2.5, 3, 3.5, 4, 4.5 and 5 and numbers in between such specific numbers).

The term “adjuvant” means a component that, when added to a dosing regimen of a single active ingredient or when added in combination with another active ingredient, such component adds or provides, in combination, an enhanced or beneficial and modified therapeutic or safety benefit to the other active ingredient in the combination when compared to the same properties of the single other active ingredient or component administered alone. The adjuvant on its own may not have clinically significant properties in the target patient population but, in combination with such other active ingredient, does provide additional therapeutic or safety clinically significant properties to such other active ingredient in the target patient population.

The terms “administering” and “administration” refer to the process by which a therapeutically effective amount of a compound or composition contemplated herein is delivered to a patient for prevention and/or treatment of the recited condition or disease.

The term “treating” refers to reversing, alleviating, or inhibiting the progression of a disease, or one or more symptoms of such disease to which such term applies. Depending upon the condition of the patient or subject, the term also refers to preventing a disease, and includes preventing the onset of such a disease depending upon the particular disease or condition.

The terms “subject” or “patient” are used interchangeably herein and include mammalian subjects including humans or animals such as horses, dogs, cows, cats or other mammals.

The term “pharmaceutically acceptable excipient or carrier” refers to a medium which does not interfere with the effectiveness or activity of an active ingredient and which is not toxic to the subjects to which it is administered. Excipients can include diluents, binders, adhesives, lubricants, disintegrates, bulking agents, wetting or emulsifying agents, pH buffering agents and other known pharmaceutically effective excipients.

The term “combination treatment” or “co-administration” means the active ingredients are administered concurrently to a patient being treated. When administered concurrently, each component may be administered at the same time, or sequentially in any order at different points of time. This term is inclusive of pre-treatment with one active ingredient and then treatment with both active ingredients and/or either active ingredient at the same or at different points in time-all in order to achieve the desired therapeutic and/or beneficial effect. Beneficial effects include, for example, a diminishment in side effects of one or both active ingredients due to the presence of the other active ingredient.

The term “a beneficial effect” refers to an effect of a compound or adjuvant or composition or combination which includes favorable pharmacological and/or therapeutic effects, and/or improved biological activity and which includes or can include a reduction in side effects. The term “beneficial effects” is inclusive of such effects as improved cognitive function, reduced vascular load, reduced astrogliosis, reduced amyloid burden, reduced microgliosis and/or improved survival. The beneficial effects may also include enhanced stability, a longer half-life, and/or enhanced uptake and transport across the blood brain barrier by one active ingredient or adjuvant to/for the overall benefit of/from the other active ingredient. Reduction of ARIA is considered to be a beneficial effect.

Immunotherapy for Alzheimer's disease is a promising approach to reduce amyloid beta fibrils and plaques in the brain. Previous clinical trials investigating active or passive immunotherapeutic approaches to lower cerebral amyloid-β burden have shown some benefit in reducing amyloid beta and improvement in cognition.¹⁻⁴ However, doses of antibody therapy used were limited by the appearance of treatment-related abnormalities on brain imaging. Although these imaging abnormalities can be clinically silent, their long-term implications on safety is uncertain and potentially dangerous.

Imaging abnormalities associated with immunotherapy have been observed in several humanized monoclonal antibody therapies against beta amyloid, including phase 2 bapineuzumab studies. [symbol] These MRI abnormalities were initially referred to as “vasogenic edema” [symbol] . As the number of studies and finding were identified in subsequent trials with most other immunotherapies, it became clear that there was a spectrum of imaging alterations associated with amyloid modifying treatments. The Amyloid-Related Imaging Abnormalities (ARIA), [symbol] which includes FLAIR signal abnormalities thought to represent parenchymal vasogenic edema and sulcal effusions (ARIA-E), as well as abnormalities detectable on GRE/T2* sequences believed to represent micro hemorrhages and hemosiderosis (ARIA-H).

The prevalence and severity of ARIA correlates closely with an increase in dosing of antibodies to amyloid beta, thus preventing the full efficacy of immunotherapy treatments by liming the administration of high levels of antibody required to optimize the reduction in brain amylolid and improve cognition in Alzheimer's disease. In most cases, immunotherapy treatments have been using treatments with suboptimal doses of amyloid beta antibodies in order to prevent the appearance of ARIA in patients.

More recently, an immune therapy to amyloid beta (aducanumab) was recently shown to be effective at higher doses of antibody administered to MCI and mild AD patients relative to the lower doses. In order to reduce the ARIA associated with higher doses required for optimal efficacy, a dose escalation regiment was used reduce the prevalence and severity of ARIA, aducanumab was administered to patients with low doses such as 1 mg/kg at first, and then increased slowly with a period of time in between to 3, 6 and then 10 mg/kg doses. The finding that by increasing the doses slowly, higher doses of the therapy could be administered resulted in efficacy with an acceptable safety profile.

Another recently approved mononclonal antibody, lecanemab, also was found to treat Alzheimer's patients but the prescribing information and label requires monitoring the patients being treated for ARIA related events using MRI both prior to and during the multi-weeks treatment course.

There is, thus, a significant unmet medical need for an agent, drug or adjuvant having particular properties that can allow/permit an increase in the dosage/dosing regimen of an immunotherapeutic agent such as aducanumab or lecanemab and other effective monoclonal antibodies by reducing/mitigating the safety issues associated with ARIA while permitting/achieving a significant increase in the dosage and efficaciousness of such immunotherapy in patients having MCI and mild AD and in need of treatment thereof. Similarly, there is a need for an adjuvant or drug that could also treat Alzheimer's patients having mild disease or subjects having MCI simultaneously with the treatment provided by the immunotherapeutic while also permitting either lower doses of the immunotherapeutic or higher doses of the immunotherapeutic drug to minimize or reduce ARIA associated events.

That need is filled by the surprising discovery that a small molecule—e.g., scyllo inositol—is able to effectively work with the monoclonal antibody in its attack on Aβ to effectively treat the Alzheimer's patients even better than aducanumab or lecanemab alone or is able to reduce the final titration strength and/or increase the strength and dose of aducanumab or lecanemab to have the same or diminished level of ARIA.

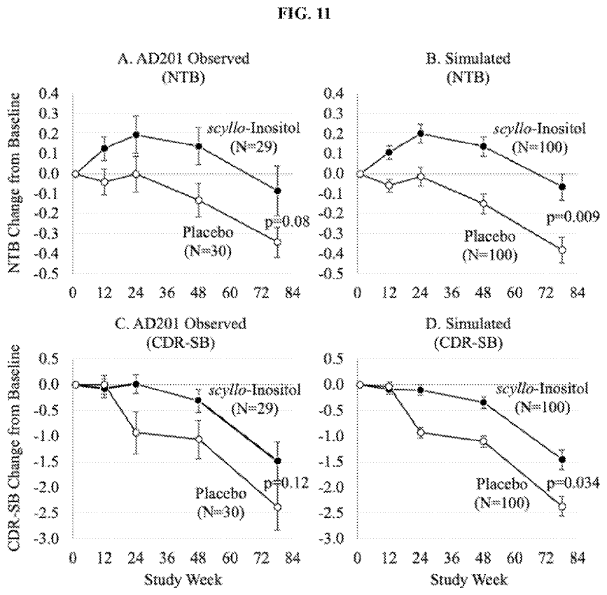

It is believed that the combination therapy of scyllo-inositol and an immunotherapeutic agent would be particularly efficacious in a sub-population of mild AD patients having MMSE scores of between 22 to 26. A comparison of observed versus simulated data for changes in NTB and CDR-SB scores with scyllo-inositol treatment of mild AD patients with such scores provided statistically significant improvement in NTB and CDR-SB. See FIG. 11.

Although the exact mechanism for the role of immunotherapy in developing ARIA is unknown, there is a possibility that increased antibody binding to large amyloid beta aggregates in the perivascular cuffs and accessible plaques in the brain result in the cause of local inflammation and leakage. High doses of antibodies to amyloid beta are required to break down these aggregates and increase a shift in the clearance of amyloid beta from the brain to the CSF and blood. As the doses of antibody are increased, the likelihood of creating pockets of antibodies reacting with amyloid aggregates and plaques increase, resulting in symptoms related to ARIA. Therefore, agents which can interact and disaggregate amyloid aggregates may have the potential to reduce these pockets of antibody complexes in the brain.

Scyllo-inositol, a stereoisomer of myo-inositol, was found to break down and prevent the formation of amyloid beta fibrils in vitro. In vivo, the daily administration of 0.3 to 30 mg/kg of scyllo-inositol to a transgenic mouse model for AD showed a decrease in amyloid beta burden in brain and an improvement of cognition and function testing. Further, treatment with scyllo-inositol showed a reduction in neurotoxicity and brain inflammation. The ability of scyllo-inositol to cross the blood brain barrier through the myo-inositol transporters allows the drug to achieve sufficient levels to reduce large amyloid aggregates and plaques to small oligomers of amyloid beta. The improvement in cognition may reflect the decrease in amyloid burden and reduction on large aggregates and plaques.

Preclinical studies were conducted as disclosed in, for example, US2007/0197452, using methods that tested Alzheimer's disease mouse models such as TgCRND8 mice. The tests conducted include behavioral tests such as Morris Water maze testing; cerebral amyloid burden; plasma and cerebral Aβ content; gliosis quantitation; survival census; analysis of APP in brain; soluble Aβ oligomer analysis; long-term potentiation; and synaptophysin immunohistochemical staining. The results obtained in such studies demonstrated the effectiveness of scyllo-inositol in treating TgCRND8 mice having morphology, density and distribution of amyloid plaques similar to those seen in the brains of human patients having Alzheimer's disease. Levels of scyllo-inositol in CSE at doses of 250 mgs BID ranged from about 10-20 ug/ml while levels of scyllo-inositol in the plasma at such doses ranged from about 5-8 ug/ml, thus meeting targeted doses necessary to break down Aβ aggregates.

The focus of this disclosure is three fold; pretreatment as well as ongoing treatment with scyllo-inositol in combination with amyloid beta antibody therapies such as aducanumab or lecanemab will reduce the prevalence and severity of ARIA, and thus allows an increase in doses of antibody administered to MCI and mild AD patients resulting in improved cognition and function; secondly, the co-administration of scyllo-inositol with antibody therapies to amyloid beta will increase the shift in clearance of amyloid beta from the brain to CSF and blood, resulting in decreased amyloid burden in the brain resulting in improved cognition relative to antibody therapy alone, lastly; co-administration of scyllo-inositol and anti-amyloid beta antibodies will enhance the efficacy in cognition and function compared to the antibody therapy alone by acting synergistically or in combination to prevent amyloid beta accumulation in the brain.

Table 1 shows a comparison of scyllo-inositol phase 2 efficacy data with Phase 2 or 3 data for donanemab, aducanamab and lecanemab.

TABLE 1

| Common | Scyllo- | | | |
|---|---|---|---|---|
| Endpoint | Inositol | Aducanumab | Lecanemab | Donanemab |
| CDR-SB | −0.95 (40%) | −0.39 (22%) | −0.45 (27%) | −0.36 (23%) |
| ADCS-ADL | −2.25 (31%) | — | −2.0 | −1.25 (24%) |
| MMSE | −1.18 (19%) | 0.6 | — | −0.6 (20%) |

The table shows the difference between active and placebo (% drug effect). The data shows that scyllo-inositol demonstrated comparable or improved efficacy in the CDR-SB test containing both cognition and functional domains when compared with the three separate immunotherapies. Scyllo-inositol also demonstrated comparable or improved efficacy in the ADCS-ADL (functional test) and MMSE when compared with donanemab data. No ARIA E or H was observed with scyllo-inositol, whereas 22-40 ARIA was observed with the immunotherapies.

The present invention comprises the use of scyllo-inositol in combination with an immunotherapeutic wherein scyllo-inositol performs at least one of the following comprising (i) breaking down Aβ fibrils; (ii) preventing Aβ binding to fibrils; (iii) increasing soluble Aβ levels in brain interstitial fluid; and (iv) increases microglia uptake of Aβ; (v) decreases Aβ burden and at an effective concentration of about 5 to 10 μM to increase Aβ clearance in a subject in need of treatment thereof.

The present invention further comprises the combination of scyllo-inositol and an immunotherapeutic selected from the group consisting of donanemab, aducanumab or lecanemab to improve cognitive function in a subject in need of treatment thereof. It is believed that such combinations are surprisingly more than additive while also beneficial in terms of reducing ARIA related events. The beneficial effects of disruption and clearance of Aβ fibrils combined with the mutual therapeutic benefits of scyllo-inositol and said immunotherapeutic with reduced ARIA side effects provides the subject with a synergistic effect in the treatment of Alzheimer's disease; especially mild disease and/or mild cognitive impairment in subjects having MMSE scores of 22 to 26.

The present invention comprises combination therapy comprising scyllo-inositol and an immunotherapeutic wherein said combination enhances Aβ clearance relative to either drug administered alone while concurrently reducing ARIA and improving cognitive function in patients in need of treatment thereof. The synergistic improvement results from scyllo-inositol's ability to penetrate the blood brain barrier and break down Aβ aggregates to produce soluble Aβ that is cleared through the microglia and CSF while, concurrently, the immunotherapeutic draw out or capture soluble Aβ from the brain into the CSF to reduce the levels of Aβ in the brain. The combination results in enhanced clearance which leads to improved cognition and function in the subjects treated with said combination(s). In addition, scyllo-inositol can solubilize Aβ aggregates in the vascular drainage pathways and basement membrane, thus reducing the formation of immune-complexes and associated ARIA.

The present invention comprises a method of reducing the severity and/or prevalence of ARIA in subjects treated with an immunotherapeutic by administering a pharmaceutically effective amount of scyllo-inositol to said subject before and concurrently with the administration of said immunotherapeutic. In a further embodiment, the invention comprises a method of disrupting the aggregation and formation of Aβ deposits in vascular drainage pathways in a subject in need of treatment thereof comprising the administration of a combination of scyllo-inositol and an immunotherapeutic selected from the group consisting of donanemab, aducanumab or lecanemab.

The invention comprises a method of reducing arterial cerebral amyloid angiopathy in a subject comprising the administration of a pharmaceutically effective amount of scyllo-inositol in combination with an immunotherapeutic in the subject wherein the reduction in said arterial cerebral amyloid angiopathy is relative to treatment in said subject with the immunotherapeutic alone.

The present invention comprising pretreatment with scyllo-inositol for 2 weeks followed by co-administering scyllo-inositol in combination with anti-amyloid beta antibody therapies to MCI and mild AD patients reduces the safety concerns associated with ARIA and enhance the reduction in amyloid beta burden in the brain resulting in improved efficacy and safety. Alternatively, patients already on monoclonal antibody therapy may be co-administered scyllo-inositol to treat MCI and mild AD. The initial clinical protocol will, however, require pretreatment for the two-week course. MCI and mild AD patients are distributed between 3 cohorts and treated as described below:
a. Cohort 1: Patients will be treated with 250 mg BID or 500 mg QD of scyllo-inositol alone for 54 weeks. The patients will be examined for amyloid burden, memory, cognition and functional tests, as well as ARIA.
b. Cohort 2: Patients will be treated with 250 mg BID or 500 mg QD of scyllo-inositol alone for two weeks, the followed a combination of 250 mg BID or 500 mg QD of scyllo-inositol and escalating doses of monoclonal antibody beginning with 1 mg/kg (4 weeks), then 3 mg/kg (4 weeks), then 6 mg/kg (4 weeks) and then a final dose of 10 mg/kg for the remaining period of the study (40 weeks). The patients will be evaluated for ARIA, amyloid beta burden, memory, cognition and function, and safety parameters.
c. Cohort 3: Patients will be treated with escalating doses of monoclonal antibody alone, beginning with 1 mg/kg (4 weeks), 3 mg/kg (4 weeks), 6 mg/kg (4 weeks) and 10 mg/kg for the remaining period of the study (40 weeks). The patients will be evaluated for ARIA, amyloid beta burden, memory, cognition and function, and safety parameters.

The combination treatment of scyllo-inositol and the immunotherapeutic for a period of 52 weeks will result in:

Decreased ARIA in comparison to treatment with immunotherapy alone.

Decreased amyloid beta burden in the brain compared to treatment of immunotherapy alone.

Improved memory, cognition and function in comparison to treatment of immunotherapy alone.

Improvement in amyloid beta biomarkers in the CSF such as amyloid beta 42/40 ratio, Tau and phosphorylated Tau.

This combination will also permit more flexibility in the dosing regimen of immunotherapeutic by permitting the modification of both the strength of the dose and by permitting a modified titration regimen of the currently approved treatment course.

An alternative phase 2 study combining scyllo-inositol and an immunotherapeutic for the treatment of MCI and Early Mild AD may also be conducted. A subset of patients having mild AD with MMSE scores ranging from either 22-26 or with scores ranging from 23-26 are recruited for the clinical trial. The number of subjects in each arm includes at least 45 to 50 patients. Three arms include (1) said patients are treated with placebo for four weeks followed by the selected immunotherapeutic for sixteen (16) weeks; (2) said patients are treated with 250 mgs BID scyllo-inositol for four weeks followed by sixteen (16) weeks with 250 mgs BID scyllo-inositol and the prescribed dosing amount and dosing frequency of the selected immunotherapeutic and (3) said patients are treated with 500 mgs BID of scyllo-inositol for four weeks followed by sixteen (16) weeks of a combination of 250 mgs BID of scyllo-inositol and the prescribed dosing amount and frequency of the selected immunotherapeutic. The primary endpoint measure is the incidence of ARIA E and H at 0, 4, 12 and 20 weeks. The secondary endpoints include the measurement of Aβ burden (PET scan) at 0, 12 and 20 weeks with other endpoints inclusive of NTB, cDR-SB, MMSE. Biomarkers such as tau and P-tau may be measured as well. The incidence or prevalence of subjects having ARIA in the immunotherapeutic treated patients is assumed to be about 22-30% ARIA E and H.

### Scyllo-Inositol

Scyllo-inositol may be obtained from processes disclosed in a number of patents and applications. See U.S. Pat. Nos. 8,409,833 and/or 7,745,671, both incorporated by reference herein. Its use in preventing, treating and diagnosing disorders of protein aggregation is disclosed in, for example, EP1608350B1 or U.S. Pat. No. 8,859,628 or 7,521,481 which are incorporated by reference herein. The data shown therein demonstrated that scyllo-inositol treatment in mice significantly decreased amyloid burden and gliosis. Scyllo-inositol is described as having the property of inhibiting already established amyloid deposition in the living brain. Thus, the data suggests that scyllo-inositol has the properties, in mammals in need of treatment thereof, of reducing amyloid plaque burden and improving cognition. Diseases that can be treated with scyllo-inositol include conditions of the central or peripheral nervous system or systemic organs and which have, as a condition, deposits of proteins or protein fragments and peptides in beta-pleated sheets and/or fibrils or aggregates. This deposition and/or tissue already having such sheets is, in a patient being treated with or prescribed a monoclonal antibody treatment for such diseases, can be interrupted by the co-administration of or combination of a suitable dosage form of scyllo-inositol and such monoclonal antibody.

Specific diseases and conditions treatable with such a combination treatment is inclusive of Alzheimer's disease, presenile and senile forms; amyloid angiopathy; mild cognitive impairment (MCI), Alzheimer's disease-related denential; tauppathy; alpha-synucleinopathy; Parkinson's disease; amyotrophic lateral sclerosis; motor neuron disease; spastic paraplegia; Huntington's disease, spinocerebellar ataxia, Freidrich's ataxia; neurodegenerative diseases associated with intracellular and/or intra-neuronal aggregates of proteins with polyglutamine, polyalanine or other repeats arising from pathological expansions of tri- or tetra-nucleotide elements within corresponding genes and other diseases and disorders disclosed in, for example, U.S. Pat. No. 7,521,481 incorporated by reference herein.

Scyllo-inositol may be formulated into any suitable pharmaceutical formulation. The compound may be delivered orally or by other suitable means. Oral formulations may be in the form of tablets or capsules which include pharmaceutically acceptable excipients selected from binders, fillers, surfactants, preservatives, lubricants and the like. The amount of the drug varies but, in the combination treatment, ranges on the higher strength side and is between 125 to 250 mgs BID or 500 mg QD. The prescribing physician may modify this dosage amount depending upon the particular disease condition or state of the patient being treated with the combination regimen and could lower the adjuvant dose amount to between 50 to 150 mgs BID. The tablets and/or capsules may be manufactured by means known to those of skill in the art. The administration of scyllo-inositol may also be accomplished by the use of oral liquids or suspensions, intraveneous administration, intramuscular administration or other means such as intraperitoneally, intradermally, transcutaneously, subcutaneously, intranasally, sublingually, inhalation or other means.

Scyllo-inositol may be formulated as an oral tablet or capsule. Tablets may be formed by compression and may be produced from crystalline, powdered or granular materials along with other pharmaceutically acceptable excipients such as binders, disintegrants, lubricants, diluents and colorants. Diluents can be selected from, for example, dicalcium phosphate, lactose, cellulose, mannitol, dry starch, powdered sugar and/or sodium chloride. Binders can be selected from starches, gelatin, and sugars such as sucrose, glucose, dextrose or lactose. Natural and/or synthetic gums may also be utilized. Lubricants such as magnesium stearate may also be incorporated into the tablet or capsule. Flavorants may also be utilized.

### Aducanumab

Aducanumab is described as a recombinant human immunoglobulin gamma 1 (IgG1) monoclonal antibody directed against aggregated soluble and insoluble forms of amyloid beta. The immunoglobulin is expressed in a Chinese hamster ovary cell line and has a molecular eight of 146 kDa. The pre-diluted injection formulation is preservative free and each mL of solution contains 100 mg of aducanumab and L-arginine hydrochloride (31.50 mg), L-histidine (0.60 mg), L-histidine hydrochloride monohydrate (3.39 mg), L-methionine (1.49 mg), polysorbate 80 (0.50 mg), and water for injection at a pH of about 5.5. The clinical studies demonstrated that ADUHELM reduces amyloid beta plaques, as described in such studies. The drug reduced amyloid beta plaque in a dose and time dependent manner compared to placebo. The effect of the drug on such plaque levels was evaluated using PET imaging (¹⁸F-florbetapir tracer). The PET signal is described as being quantified using the Standard Uptake Value Ratio (SUVR) method to estimate brain levels of amyloid beta plaque in composites of brain areas expected to be affected by Alzheimer's disease pathology. See Prescribing Information for ADUHELM. These areas are frontal, parietal, lateral, temporal, sensorimotor, and anterior and posterior cingulate cortices as compared to a brain region expected to be fee of such pathology (cerebellum).

Substudies of such clinical studies of aducanumab showed reductions in amyloid beta plaque levels in the brain at both low dose levels and high dose levels at weeks 26 and 78 compared to placebo. The magnitude of such reduction again was described as both dose and time dependent.

In a third clinical study of ADUHELM, statistically significant dose and time dependent reductions of amyloid plaque levels was shown in the 3 mg/kg, 6 mg/kg and 10 mg/kg treatment groups at week 26 and in all treatment groups at week 54 as compared to placebo treatments.

ADUHELM was also studied for its effect on Tau pathophysiology (marker levels). The studies demonstrated that ADUHELM reduced markers of tau pathophysiology (CSF p-Tau and Tau PET) and neurodegeneration (CSF t-Tau) (Studies 1 and 2). The immunotherapeutic drug also reduced CSF levels of p-Tau in substudies conducted in Study 1 and Study 2. At week 78 in Study 1, the adjusted mean changes from baseline in CSF p-Tau levels relative to placebo was in favor of ADUHELM low and high dose groups. The drug also reduced CSF levels of t-Tau in substudies conducted in Study 1 in the low dose and high dose groups compared to placebo.

Sub-studies were also conducted in Studies 1 and 2 to see the effect of aducanumab on neurofibrillary tangles composed of tau protein using PET imaging (¹⁸F-MK6240 tracer). The PET signals are quantified using the SUVR method to estimate brain levels of tau in brain regions expected to be affected by Alzheimer's disease pathology (medial temporal, temporal, frontal, cingulate, parietal and occipital cortices) compared to a brain region not expected to be effected—e.g. the cerebellum. The clinical data showed that the adjusted mean change from baseline in tau PET SUVR relative to placebo at follow up was in favor of aducanumab administered at the high dose and in the medial temporal, temporal and frontal regions of the brain.

Finally, additional data was collected on exposure response relationships after taking aducanumab versus placebo. The data demonstrated that higher exposures to aducanumab were associated with a greater reduction in clinical decline of the subjects as measured by CDR-SB, ADAS-Cog13 and ADCS-ADL-MCI along with greater reduction in amyloid beta plaque.

The clinical studies conducted on the combination product recited herein will use the same methods utilized in the ADUHELM studies to show efficacy, marker decline and improvement in AIRA related events following combination treatment and pre-treatment with scyllo-inositol.

As articulated in U.S. Pat. No. 10,842,871 which is incorporated by reference herein, during the course of the development of drugs to treat Alzheimer's disease the Food and Drug Administration (“FDA”) expressed concerns in 2010 related to the development of abnormalities following treatment in clinical trials that showed up on MRI scans. These abnormalities were identified and/or thought to represent vasogenic edema (VE) and microhemorrhages (mH) and which were first observed in clinical trials of monoclonal antibodies to amyloid β. Thereafter developers of such drugs, including the developer of aducanumab, a recently approved monoclonal antibody treatment, focused on efficacy and safety and on the new concerns with respect to VE or mH that arise because of the monoclonal antibody treatment. The underlying reason for the increase in VE and mH abnormalities is not fully understood. The presence of Apolipoprotein E ε4 allele, ApoE ε4, has been found to be a significant risk factor for the development of ARIA-E (VE associated MRI abnormalities). mH, on the other hand, were not found to be associated with any particular allele but are generally attributed to one of two etiologies; small vessel angiopathy and cerebral amyloid angiopathy (CAA). It has also been suggested that focal inflammatory components due to drug treatment may cause both ARIA-E and/or ARIA-H.

In any case, in order to both treat MCI and mild Alzheimer's disease and mitigate ARIA arising from such treatment, Biogen-IDEC secured FDA approval of ADUHELM™ aducanumab having a treatment regimen which requires titration in order to mitigate against ARIA. The drug is an amyloid beta-directed antibody indicated for the treatment of Alzheimer's disease and was approved under accelerated approval based upon the reduction in amyloid beta plaques observed in patients treated with ADUHELM. The Dosage and Administration section of the approved label provides that (1) titration is required for treatment initiation; (2) The recommended maintenance dosage is 10 mg/kg administered as an intravenous infusion over approximately one hour every four weeks; (3) a recent (within one year) brain MRI prior to initiating treatment; (4) obtain MRIs prior to the 7^{th} an 12^{th} infusions. If radiographic severe ARIA-H is observed, treatment may be continued with caution only after a clinical evaluation and a follow-up MRI demonstrates radiographic stabilization (i.e., no increase in size or number of ARIA-H); (5) dilution t 100 mL of 0.9% sodium chloride injection USP, prior to administration; and (6) administer as an intravenous infusion over approximately one hour via a 0.2 or 0.22 micron in-line filter. The approved dosage forms and strengths are injection 170 mg/1.7 mL (100 mg/mL) solution in a single-dose vial and 300 mg/3 mL (100 mg/mL) solution in a single dose vial. The Warnings and Precautions section of the label warns that amyloid related imaging abnormalities (ARIA) require enhanced clinical vigilance for such ARIA during the first 8 doses of treatment, particular during the titration period. The period between IV infusions is every four weeks. The dosing or titration schedule for infusions 1 and 2 is 1 mg/kg aducanumab; for infusions 3 and 4 is 3 mg/kg; for infusions 5 and 6 is 6 mg/kg and for infusion 7 and beyond 10 mg/kg. The Adverse Reactions section of the label states that the most common adverse reactions (at least 10% and higher incidence compared to placebo) are ARIA-Edema, headache, ARIA-H microhemorrhage, ARIA-H superficial siderosis, and fall.

The Monitoring for ARIA section states that if 10 or more new incident microhemorrhages or greater than 2 focal areas of superficial siderosis (radiographic severe ARIA-H) is observed, treatment may be continued with caution only after a clinical evaluation and a follow-up MRI demonstrates radiographic stabilization (i.e., no increase in the size or number of ARIA-H).

In clinical studies of ADU HELM and in clinical studies of the combination recited in this specification, the severity of ARIA is classified by radiographic criteria as provided in Table 2 below:

TABLE 2

| ARIA | | | |
|---|---|---|---|
| Type/ | | | |
| Severity | Mild | Moderate | Severe |
| ARIA-E | FLAIR | FLAIR | FLAIR |
| | hyperintensity | hyperintensity | hyperintensity |
| | confined to | 5 to 10 cm, or | measuring |
| | sulcus and or | more than 1 | >10 cm, often |
| | cortex/ | site of involve- | with |
| | subcortical | ment each | significiant |
| | white matter | measuring | subcortical |
| | in one | <10 cm | white matter |
| | location | | and/or sulcal |
| | <5 cm | | involvement. |
| | | | One or more |
| | | | separate sites |
| | | | of involvement |
| | | | may be noted. |
| ARIA-H | Less than or | 5 to 9 new | 10 or more |
| micro- | equal to 4 | incident | new incident |
| hemorrhage | new incident | micro- | micro- |
| | micro- | hemorrhages | hemorrhages |
| | hemorrhages | | |
| ARIA-H | 1 focal area of | 2 focal areas of | 2 focal areas |
| Superficial | superficial | superficial | of superficial |
| siderosis | siderosis | siderosis | siderosis |

In clinical studies of aducanumab treatment alone versus placebo, ARIA E and/or H was observed in 41% of patients treated with drug with a planned dose of 10 mg/kg (454 out of 1105), compared to 10% of patients on placebo (111 out of 1087). ARIA-E was observed in 35% of patients treated with aducanumab 10 mg/kg, compared to 3% of patients on placebo. As previously discussed, the incidence of ARIA-E was higher in apolipoprotein E ε4 (ApoE ε4) carries than in ApoE ε4 non-carriers (42% and 20% respectively). The clinical studies demonstrated that the majority of ARIA-E radiographic events occurred early in treatment (within the first 8 doses) although it was stated that such ARIA can occur at any time. Among the patients treated with aducanumab (10 mg/kg) and which had ARIA-E events, the maximum radiographic severity was mild in 30%, moderate in 58%, and severe in 13% of the patients. Resolution occurred in 68% of ARIA-E patients by 12 weeks, 91% by 20 weeks, and 98% overall after detection. 10% of all patients who received aducanumab 10 mg/kg had more than one episode of ARIA-E. ARIA-H in the setting of ARIA-E associated with the use of ADUHELM 10 mg/kg was observed in 21% of patients treated with drug versus 1% of patients on placebo.

ADUHELM is administered in a titration-based regimen because of the need to reduce ARIA associated events which occur or are more likely to occur in a fixed dose regimen. The titration is believed to slow the pace of initial amyloid remove and thus permit slower removal during the overall treatment of the patient. It is believed that pre-treatment with a non-monoclonal antibody regimen of, for example, a drug such as scyllo-inositol, will facilitate the removal of plaque and reduce plaque burden without causing ARIA related events so that the subsequent co-administration of scyllo-inositol and aducanumab will lower ARIA associated or related events due to antibody treatment and thus permit titration at higher doses of aducanumab and/or facilitate a fixed dose regimen of aducanumab/scyllo-inositol without having ARIA associated events or having fewer such events in the patients being treated. In the combination, the pace of plaque removal can thus be accelerated without the need to slow the removal of amyloid.

Aducanumab (BIIBO37) is an IgG₁ monoclonal antibody consisting of 2 heavy and 2 kappa light chains connected by inter-chain disulfide bonds. The antibody recognizes a conformational epitope found in Aβ aggregates. It has been disclosed that a murine IgG2a chimeric version of this antibody (chl 2F6A) lowers or reduces plaque burden in aged Tg2576 mice which is a mouse model of Alzheimer's disease. See Wilcock and Colton 2009. The human version of antibody 12F6A has an identical amino acid sequence to BIIB037 which is produced in a different Chinese hamster ovary cell line.

Aducanumab has antigen binding domains comprising V_{H} and/or V_{L} variable regions as shown in Table 3.

TABLE 3
VH
QVQLVESGGGVVQPGRSLRLSCAASGFAFSSYGMHWVRQAPGKGLEWVAV
IWFDGTKKYYTDSVKGRFTISRDNSKNTLYLQMNTLRAEDTAVYYCARDR
GIGARRGPYYMDVWGKGTTVYVSS (SEQ ID NO: 1)
VL
DIQMTQSPSSLSASVGDRVTITCRASQSISSYLNWYQQKPGKAPKLLIYA
ASSLQSGVPSRFSGSGSGTDFTLTISSLQPEDFATYYCQQSYSTPLTFGG
GTKVEIKR (SEQ ID NO: 2)

Aducanumab (BIIB037) has the following denominations of CDR protein sequences:

TABLE 4

| | Variable Heavy | Variable Light |
|---|---|---|
| CDR | Chain | Chain |
| CDR1 | SYGMH | RASQSISSYLN |
| | (SEQ ID NO: 3) | (SEQ ID NO: 6) |
| CDR2 | VIWEDGMKYYTDSVKG | AASSLQS |
| | (SEQ ID NO: 4) | (SEQ ID NO: 7) |
| CDR3 | DRGIGARRGPYYMDV | QQSYSTPLI |
| | (SEQ ID NO: 5) | (SEQ ID NO: 8) |

The heavy chain of anti-Aβ antibody BIIB037 has the following sequence:

(SEQ ID NO: 11)
QVQLVESGGG VVQPGRSLRL SCAASGFAFS SYGMHWVRQA
PGKGLEWVAV IWFDGTKKYY TDSVKGRFTI SRDNSKNTLY
LQMNTLRAED TAVYYCARDR GIGARRGPYY MDVWGKGTTV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK RVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGRY PSDIAVEWES NGQPENNYKT
TPPVLDSDGS FFLYSKLTVD KSRWQQGNVF SCSVMHEALH
NHYTQKSLSL SPG

Heavy chain CDRs are underlined.

The light chain of anti-Aβ antibody BIIB037 has the following sequence:

(SEQ ID NO: 12)
DIQMTWSPSS LSASVGDRVT ITCRASQSIS SYLNWYQQKP
GKAPKLLIYA ASSLQSGVPS RFSGSGSGTD FTLTISSLQP
EDFATYYCQQ SYSTPLTFGG GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC.

Light chain CDRs are underlined.

The antibodies can be produced using processes described in, for example, US2021018895 which is hereby incorporated by reference. As recited therein, they can be produced in eukaryotic cells or in bacterial cells. In preferred embodiments, they are produced in transformed eukaryotic cell lines such as CHO, 292E and COS. In addition to bacterial cells and eukaryotic cells, yeast cells can also be used to produce antibodies or scFvs thereof. A general process involves constructing a polynucleotide encoding the antibody, introducing this into an expression vector and expressing the antibody in a suitable host cell. Molecular biology techniques are known to those of skill in the art. Promoters such as an SV40, MMLV-LTR or EF1α or CMV promoters are required if the antibody is expressed in CHO, COS or NIH3T3 cells. Additional sequences such as regulatory sequences may be added and these can facilitate replication and selection and can confer resistance to drugs into which the vector has been introduced. Suitable vectors are inclusive of pMAM, pDR2 and others as described in US2021188954. To illustrate the production of BIIB037, a recombinant expression vector encoding the heavy and light chains of the antibody are introduced into dhfr− CHO cells by calcium phosphate-mediated transfection. The antibody heave and light chains are operative linked to enhancer/promoter regulatory elements derived from any one of SV40, CMV etc. such as a CMV enhancer/AdML: promoter regulatory element or SV40 enhancer in such a system to encourage high levels of transcription of the genes. The vectors were also made to include a DHFR gene which allows for the selection of CHO cells that have been transfected with the vector using methotrexate selection/amplification. The selected transformed cells are cultured to provide expression of the antibody light and heavy chains and the antibody is then recovered from the culture medium and used in the compositions described herein for the treatment of Alzheimer's disease patients. Purification methods are known in the art and such antibodies can be isolated and purified to levels necessary for human administration. Methods of purification include column chromatograph, filtration, ultra-filtration, salting out, solvent extraction, solvent precipitation, immunoprecipitation and other means inclusive of SDS-polyacrylamide gel electrophoresis, isoelectric focusing, dialysis and recrystallization.

Compositions of the antibody can be formulated according to the methods and composition described in U.S. Pat. No. 10,842,871, which is incorporated by reference herein. The compositions can comprise pharmaceutically acceptable excipients such as phosphate buffered saline solutions, water, emulsions including oil in water emulsions and the like. Wetting agents can be added and such compositions can be delivered as a sterile solution. The administration of the antibody and pharmaceutical composition can be by, for example, intravenous, intraperitoneal, subcutaneous, intramuscular, topical or transdermal administration. Various concentrations of the antibodies can be prepared and utilized in the combination treatment. Such concentrations can range from 50 mg/mL to over 300 mg/mL as high concentration antibody compositions. Sterile injectable solutions of such antibodies are made and require filtered sterilization. Coatings of such antibodies using lecithin may be required to ensure proper fluidity of the samples. Additional ingredients are added which can, for example, lower the risk of aggregation and/or ensure proper viscosity. Excipients can include, for example, L-arginine hydrochloride at various concentration ranges (40 to 260 nM and ranges in between). Sucrose can be additionally added at a concentration ranging from about 0.5% to about 5%. Methionine may also be included in the composition at a concentration range of 5 mM to about 150 mM. Other excipients to facilitate formulation and handling may include polysorbate at a concentration range of 0.01% to 0.03%. Buffers may also be added to achieve a pH range of about 5.0 to 6.5 or levels in between. Histidine may be utilized as a buffering agent at a concentration range of about 5 mM to 50 mM or values in between. Antioxidants such as glutathione CSH, cysteine, cystine at concentration ranges of about 0.02 mM to 4 mM may be utilized.

### Lecanemab

LEQEMBI (lecanemab) (BAN2401) is indicated for intraveneous use as an amyloid beta-directed antibody for the treatment of Alzheimer's disease. Treatment with this drug should be initiated in patients having mild cognitive impairment or mild dementia stage of disease. The warnings and precautions section of the highlights of prescribing information also refers to amyloid related imaging abnormalities (ARIA) and enhanced clinical vigilance for ARIA is recommended during the first 14 weeks of treatment with LEQEMBI. Risk of ARIA, including symptomatic ARIA, was described as being increased in apolipoprotein E ε4 homozygotes compared to heterozygotes and noncarriers. Lecanemab-irmb is a recombinant humanized immunoglobulin gamma 1 (IgG1) monoclonal antibody directed against aggregated soluble and insoluble forms of amyloid beta. It is expressed in a CHO cell line and has an approximate molecular weight of 150 kDa. It is available commercially in single-dose vials at concentrations of 500 mg/5 mL (100 mgs/mL) or 200 mg/2 mL (100 mg/mL). The solution contains histidine hydrochloride monohydrate, polysorbate, histidine, arginine hydrochloride and water at a pH of 5.0.

For LEQEMBI (lecanemab), once the presence of amyloid beta is confirmed, the recommend dosage is 10 mg/kg that must be diluted and then administered as an intravenous infusion over approximately one hour, once every two weeks. MRIs are required to assess ARIA prior to initiating treatment and prior to the 5^{th}, 7^{th} and 14^{th} infusions. Monoclonal antibodies directed against aggregated forms of beta amyloid, including LEQEMBI, are known to cause ARIA, characterized as ARIA with edema (ARLA-E) which is detectable on MRI scans as brain edema or sulcal effusions, as stated on the prescribing information. Reduction or elimination of clusters or aggregated forms of beta amyloid would reduce ARIA due to monoclonal antibody treatment.

Lecanemab comprises sequences selected from the group consisting of:

Subunit I
(SEQ ID NO: 9)
EVQLVESGGG LVQPGGSLRL SCSASGFTFS SFGMHWVRQA
PGKGLEWVAY ISSGSSTIYY GDTVKGRFTI SRDNAKNSLF
LQMSSLRAED TAVYYCAREG GYYYGRSYYT MDYWGQGTTV
TVSSASTKGP SVFPLAPSSK STSGGTAALG CLVKDYFPEP
VTVSWNSGAL TSGVHTFPAV LQSSGLYSLS SVVTVPSSSL
GTQTYICNVN HKPSNTKVDK RVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGFY PSDIAVEWES NGQPENNYKT
TPPVLDSDGS FFLYSKLTVD KSRWQQGNVF SCSVMHEALH
NHYTQKSLSL SPGK
Subunit 2
(SEQ ID NO: 9)
EVQLVESGGG LVQPGGSLRL SCSASGFTFS SFGMHWVRQA
PGKGLEWVAY ISSGSSTIYY GDTVKGRFTI SRDNAKNSLF
LQMSSLRAED TAVYYCAREG GYYYGRSYYT MDYWGQGTTV
TVSSASTKGP SVFPLAPSSK STSGGTAALG CLVKDYFPEP
VTVSWNSGAL TSGVHTFPAV LQSSQLYSLS SVVTVPSSSL
GTQTYICNVN HKPSNTKVDK RVEPKSCDKT HTCPPCPAPE
LLGGPSVFLF PPKPKDTLMI SRTPEVTCVV VDVSHEDPEV
KFNWYVDGVE VHNAKTKPRE EQYNSTYRVV SVLTVLHQDW
LNGKEYKCKV SNKALPAPIE KTISKAKGQP REPQVYTLPP
SREEMTKNQV SLTCLVKGFY PSDIAVEWES NGQPENNYKT
TPPVLDSDGS FFLYSKLTVD KSRWQQGNVF SCSVMHEALH
NHYTQKSLSL SPGK
Subunit 3
(SEQ ID NO: 10)
DVVMTQSPLS LPVTPGAPAS ISCRSSQSIV HSNGNTYLEW
YLQKPGQSPK LLIYKVSNRF SGVPDRFSGS GSGTDFTLRI
SRVEAEDVGI YYCFQGSHVP PTFGPGTKLE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC
Subunit 4
(SEQ ID NO: 10)
DVVMTQSPLS LPVTPGAPAS ISCRSSQSIV HSNQNTYLEW
YLQKPGQSPK LLIYKYSNRF SGVPDRFSGS GSGTDFTLRI
SRVEAEDVGI YYCFQGSHVP PTFGPGTKLE IKRTVAAPSV
FIFPPSDEQL KSGTASVVCL LNNFYPREAK VQWKVDNALQ
SGNSQESVTE QDSKDSTYSL SSTLTLSKAD YEKHKVYACE
VTHQGLSSPV TKSFNRGEC

Logovinsky, et al., reported on the safety and tolerability of BAN2401 in a clinical study in Alzheimer's disease with this protofibril selective Aβ antibody. See Logovinsky, et al. Alzheimer's Research & Therapy (2016) 8:14. Protofibrils are soluble Aβ aggregates, which are larger than about 100 kDa. The article states that there is increasing evidence that soluble oligomers and protofibrils are more toxic than insoluble fibrils. Safety and tolerability were assessed in mild to moderate Alzheimer's subjects. The study used staggered parallel single and multiple ascending doses of BAN2401 from 0.1 mg/kg as a single dose to 10 mg/kg per dose biweekly for four months. The presence or absence of ARIA (ARIA-E and ARIA-H) was measured using MRI and cerebrospinal fluid (CSF) and plasma samples were also analyzed to determine biomarker pharmacokinetics and possible drug effects on such markers. The data essentially showed that BAN2401 was well tolerated and that the incidence of ARIA-E/measured by MRI was comparable to placebo. The results permitted movement into a Phase 2b efficacy study.

BAN2401 is described as the humanized IgG1 monoclonal version of mAb158, a murine monoclonal antibody, that selectively binds to Aβ protofibrils. U.S. Pat. No. 8,025,878 discloses an isolated antibody or fragment thereof being selective and having high affinity for human Aβ protofibrils wherein the antibody or fragment in its six CDR regions comprises the consensus sequences selected from:

VH-CDR-1
SEQ ID NO: 13
SFGMH
VH-CDR2
SEQ ID NO: 14
YISSGSSTIYYGDTVKG
VH-CDR3
SEQ ID NO: 15
EGGYYYGRSYYTMDY
VL-CDR1
SEQ ID NO: 16
RSSQSIVHSNGNTYLE
VL-CDR2
SEQ ID NO: 17
KVSNRFS
VL-CDR3
SEQ ID NO: 18
FQGSHVPPT.

U.S. Pat. No. 9,573,994 discloses an antibody or antigen binding fragment thereof having affinity against Aβ protofibrils, wherein the antibody or antigen binding fragment thereof has a variable light chain according to SEQ ID NO: 8 therein (SEQ ID NO: 19 herein) wherein:
x1 (X at position 17 of SEQ ID NO: 19) is selected from A, D, E and Q, or a functional analogue thereof;
x2 (X at position 79 of SEQ ID NO: 19) is selected from R, T, K, A and G. or a functional analogue thereof,
x3 (X at position 82 of SEQ ID NO: 19) is selected from R, S, C g, and N, or afunctional analogue thereof;
y1 (X at position 13 of SEQ ID NO: 19) is selected from V and A;
y2 (X at position 21 of SEQ ID NO: 19) is selected from I and V;
y3 (X at position 81 of SEQ ID NO: 19) is selected from S and Q;
y4 (X at position 84 of SEQ ID NO: 19) is selected from E and D; and optionally,
a variable heavy chain according to SEQ ID NO: 14 therein (SEQ ID NO: 20 herein) wherein
z1 (X at position 37 of SEQ ID NO: 20) is selected from V and 1;
z2 (X at position 38 of SEQ ID NO: 20) is selected from R and Q; and
z3 (X at position 40 of SEQ ID NO: 20) is selected from A, N and T;
with the exception for the combination x1=A, x2=R and x3=R.

SEQ ID NO: 19:
DVVMTQSPLSLPXTPGXPAS
XSCRSSQSIVHSNGNTYLEW
YLQKPGQSPKLLIYKVSNRF
SGVPDRESGSGSGTDETIXI
XXVXAEDVGIYYCFQGSHVP
PTFGPGTKLEIK
SEQ ID NO: 20
EVQLVESGGGLVQPGGSLRL
SCSASGFTFSSFGMHWXXQX
PGKGLEWVAYISSGSSTIYY
GDTVKGRFTISRDNAKNSLF
LQMSSLRAEDTAVYYCAREG
GYYYGRSYYTMDYWGQGTTV
TVSS

SEQ ID NO:12 (light chain) and SEQ ID NO: 16 (heavy chain) as disclosed in U.S. Pat. No. 9,573,994 (SEQ ID NOS: 21 and 22 herein) are almost identical or are identical to SEQ ID NOS: 10 and 9 respectively disclosed herein.

SEQ ID NO: 21
DVVMTQSPLSLPATPGDPAS
ISCRSSQSIVHSNGNTYLEW
YLQKPGQSPKLLIYKVSNRF
SGVPDRFSGSGSGTDFTLTI
SRVDAEDVGIYYCEQGSHVP
PTEGPGTKLEIK
SEQ ID NO: 22:
EVQLVESGGGLVQPGGSLRL
SCSASGFTFSSFGMHWVRQT
PGKGLEWVAYISSGSSTIYY
GDTVKGRFTISRDNAKNSLF
LQMSSLRAEDTAVYYCAREG
GYYYGRSYYTMDYWGQGTTV
TVSS

Swanson, et al. reported on a randomized, double-blind, phase 2b proof of concept clinical trial in early Alzheimer's disease with lecanemab. See Swanson, et al. Alzheimer's Research and Therapy (2021) 13:80. Lecanemab (BAN2401) was described as preferentially targeting soluble aggregated amyloid beta (Aβ), with activity across oligomers, protofibrils, and insoluble fibrils. The primary endpoint in the study was described as a Bayesian analysis of 12-month clinical change on the Alzheimer's Disease Composite Score (ADCOMS) for the ED90 dose, and which required an 80% probability of ≥25% reduction in decline versus placebo. Secondary endpoints included 18-month Bayesian and frequentist analyses of brain amyloid reduction using positron emission tomography; clinical decline on ADCOMS, clinical Dementia rating-Sum-of-boxes (CDR-SB), and Alzheimer's Disease Assessment Scale-Cognitive Subscale (ADS-Cog14); changes in CSF core biomarkers; and total hippocampal volume (HV) using volumetric magnetic resonance imaging. The results of the study showed the drug did not meet the primary endpoint but phase 3 studies were initiated because of the promising results in this phase 2 b study which showed prespecified 18-month Bayesian and frequentist analyses demonstrated reduction in brain amyloid accompanied by a consistent reduction of clinical decline across several clinical and biomarker endpoints as well as a low incidence (9.9%) of amyloid-related imaging abbnoralities-deema/effusion at the 10 mg/kg biweekly dose of lecanemab. The breakdown between ApoE4 positive patients having ARIA-E versus patients being ApoE4 negative was 14.3% versus 8.0%, respectively, with the average being 9.9%.

Lecanemab was approved for use in 2023. Notably, the patients enrolled in the clinical study conducted prior to approval (i.e., Study 1), had Clinical Dementia Rating (CDR) global score of 0.5 or 1.0 and a Memory Box score of 0.5 or greater. In addition, all patients had a Mini-Mental State Examination (MMSE) score of ≥22. It is believed that the combination of scyllo-inositol and lecanemab in the same patient population would result in a significant improvement in the primary endpoints of these studies and would result in a significant reduction in ARIA-E events in both ApoEE4 positive and negative subjects.

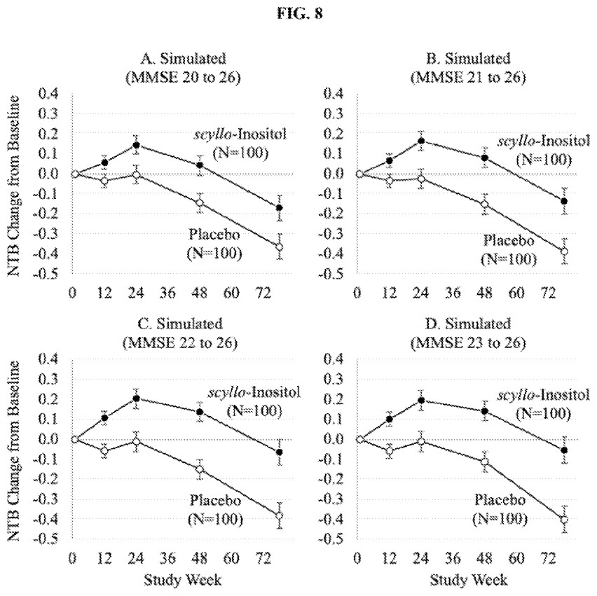

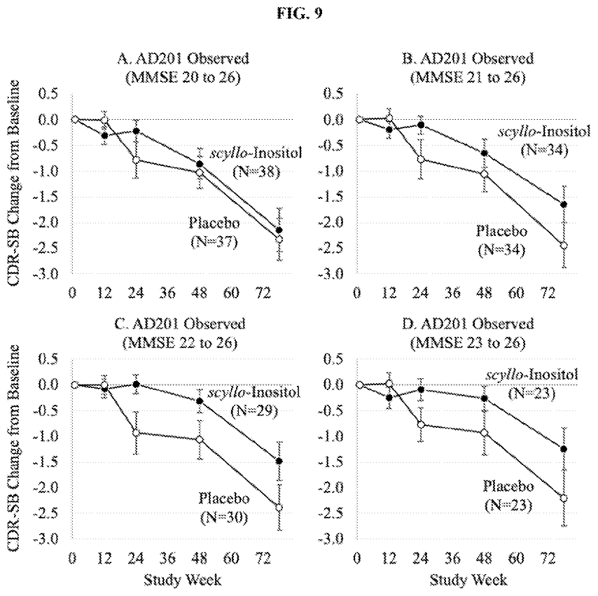

U.S. Pat. No. 8,025,878 discloses the murine antibody that is the lead murine antibody to lecanemab or the humanized monoclonal antibody, both of which are selective for amyloid beta protein (Aβ) in its protofibril conformation and of IgG class and IgG1 or IgG4 subclass or combinations thereof. The Figures and detailed examples in this patent provide the characterization data; therapeutic efficacy in a transgenic mouse model and the reactivity of mAb158 with Aβ protofibrils versus Aβ fibril, medin fibril, iset amyloid polypeptide (IAPP) fibril, and α-synuclein fibril in dot blot assays. The data in this patent also provides immunoprecipitation data from HEK-cell culture media along with data showing sandwich ELISA results using mAb158 as the capturing antibody and detecting antibody. FIG. 8 in the disclosure provides the Aβ protofibril levels in APP Arc-Swe and APP Swe transgenic mice after four months treatment with mAb158 or placebo. U.S. Pat. No. 8,025,878 also discloses the humanized antibody BAN2401 (lecanemab) binding activity and the results showed it had identical binding properties as mAb158. Example II provides the production methods to produce the chimeric antibody BAN2401. Example 8 therein provides the methods utilized to demonstrate in vivo activity in transgenic mice. mAb158 (12 mg/kg) was injected i.p. once weekly for 18 weeks in 9-10 months old APPs weare mice. When the study was completed, the mice brains were isolated and homogenized in TBS and centrifuged to sediment insoluble material. The insoluble material was then solubilized in formic acid. Two fractions were thus obtained from the mouse brains—a TBS fraction and a formic acid fraction. Aβ protofibril levels in the TBS fractions were determined using ELISA. The example showed a significant reduction of Aβ protofibril levels in the mAb158 treated mice relative to the placebo group. FIG. 8 in this patent shows the results. Total Aβ in the formic acid fraction was also determined by ELISA noting that the formic acid was utilized to solubilize all Aβ forms, in order to make all such forms detectable. FIG. 9 in this patent shows the results which demonstrated a significant reduction of total Aβ in the drug treat mice relative to the control group. These same studies can be conducted using combinations of any of the monoclonal antibodies, but more particularly those that show selective affinity for Aβ protofibrils such as lecanemab or mAb158 in mice studies in combination with scyllo-inositol to determine the effect in transgenic mice of the combination treatment(s). The studies can include mice that are pre-treated with scyllo-inositol for a period of time and/or can be done with treatment of scyllo-inositol and the monoclonal antibody at the same time points and for the same duration of treatment.

U.S. Pat. No. 9,573,994 discloses and claims Aβ protofibril binding antibodies including BAN2401 (lecanemab). This patent discloses that the humanized form of mAb1158 known as BAN2401 has enhanced properties such as prolonged half-life if certain mutations are introduced into certain positions of the variable light chain of BAN2401. These are inclusive of Kabat positions 17, 74 and 77 of such variable light chain. This mutated version is now known as lecanemab. The A, R, R at these respective positions on BAN2401 variable light chain are modified as recited in this patent which is incorporated by reference herein in its entirety. Example 5 shows cx vivo whole protein T cell assay of BAN2401 relative to the mutated forms A17D, A17D/R79T and A17D/R79T/R82S. The results showed the risks of immunogenicity was low for BAN2401 and borderline low for A17D while A17D/R79T and A17D/R79T/R82S revealed unexpectedly higher risks of immunogenicity.

The '994 patent discloses these antibodies for use in treatment or prophylaxis of Alzheimer's disease and other disorders associated with Aβ protein aggregation. Such particular uses are inclusive of traumatic brain injury (TBI), Lewy body dementia (LBD), Downs syndrome, amyotrophic lateral sclerosis (ALS), frontotemporal dementia, tauopathies, systemic amyloidosis, atherosclerosis and Parkinsons's dementia. The methods include use of the antibodies and antibody binding fragments thereof.

### Donanemab

Donanemab is a humanized IgG1 antibody which targets N-terminal pyroglutamate Aβ epitope that is known to be located on established plaques. The monoclonal antibody has no off-target binding to other Aβ species. The phase 2 studies reported in the New England Journal of Medicine and other journals included subjects that had early symptomatic Alzheimer's disease defined as prodromal Alzheimer's disease in which mild cognitive impairment is apparent or subjects with mild Alzheimer's disease with dementia. The trial included subjects having MMSE scores ranging between 20 to 28. This clinical trial is known as the TRAILBLAZER-ALZ trials. U.S. Pat. No. 8,679,498 discloses and claims donanemab and is hereby incorporated by reference in its entirety. This patent describes and discloses ex vivo target engagement studies which described immunohistochemical analyses on exogenously added Aβ antibodies to determine ex vivo target engagement on brain sections from a fixed PDAPP transgenic mouse brain (24 months old). This mouse model has been described as developing much of the pathology of Alzheimer's disease or associated therewith. Biotin tags were used on the murine antibodies utilized in this example which was conducted on murine tissue. The biotinylated 3D6 N terminal (1-5) antibody in such studies was described as robustly labeling substantial quantities of deposited Aβ in the PDAPP hippocampus. Similarly, the examples in this patent disclosure demonstrate in vivo target engagement in the mouse models and also describe therapeutic plaque lowering studies in 23-month-old PDAPP mice.

These therapeutic plaque lowering studies were conducted as follows: Negative control antibody (IgG2a), along with 3D6, mE8 (IgG1), and mE8c 9IgG2a) were injected subcutaneously at a dosage of 12.5 mgs/kg weekly for three months. A group of mice was necropsied at the start of the study (time zero) to determine the initial plaque load at 23-months of age. At the end of the study, plasma was obtained and the brains were processed for biochemical and histological outcomes (one hemi-brain each). The hippocampus and conical regions were then homogenized in 5M guanidine and the Aβ content was measured by acid urea gels followed by Western blotting. The data in the patent showed that the control group had a significant non-increase in deposited Aβ plaque load thereby confirming that the mice were at their plaque plateau. Treatment with the comparator antibody 3D6 had no effect on plaque lowering. The treatment with the N3pGlu antibodies mE8 or mE8c resulted in significant plaque lowering as comparted to the IgG2a negative control antibody. The mE8 and mE8c lowered the hippocampal Aβ1-42 levels by about 38% and 53% respectively.

Similar studies can be conducted with any of the monoclonal antibodies described herein or murine versions thereof to measure therapeutic plaque lowering effects of the combination of a monoclonal antibody with scyllo-inositol.

Methods utilized to measure clinical efficacy and outcomes are determined on a patient-by-patient basis and which involve measuring and determining the existence, severity and progression of Alzheimer's disease over a period of time. This involves clinically determining the global functioning level of the patient; daily living behavior and deficits in living capacity or behavior; volumetric analysis of brain structures and in vivo measurements of disease related deposits of abnormal proteins in the brain using techniques such as PET imaging for beta amyloid proteins. In addition, the measurement of blood, body fluid or CSF markers as an indicator of the presence of disease or progression of disease is also performed and includes measurement of tau proteins and other biomarkers such as pyroglutamate-Aβ, Aβ40 and Aβ42 in blood as well as total Tau, phosphor Tau, pyroglutamate-Aβ, Aβ40 and Aβ42 in CSF. ApoE isotype as well as hippocampal volumetric (HCV) MRI are useful as well to define and/or stage disease progression. The measurement of such markers and methods to determine such marker levels are known in the art. In addition, it is known that such markers can be predictive of pathogenesis in Alzheimer's disease. See, for example, Duyckaerts (2011) Lancet Neurol. 10, 774-775. And Craak, et al., (2013), Acta Neuropath., 126:631-41.

Amyloid plaque burden is measured by 18F-AV-45 PET. 18F-AV-45 is a known amyloid ligand sold and developed by Avid Radiopharmaceuticals. The skilled PET imaging specialist can review the PET images obtained to determine the mean uptake of 18F-AV-45 in and between AD patients and age-matched control subjects. PET measurements of regional glucose metabolism and morphometric MRI measurements are also utilized to assess AD state or progression. MRI is monitor ARIA related events.

Clinical assessments employed to determine the stages of Alzheimer's disease and overall progression and/or improvement in stopping or ameliorating disease progression use CDR, FCSRT, Neuropsychiatric Inventor-Questionaire (NPI-Q), and neurological test batteries which include Rey Auditory Verbal Learning Test (RA VLT), immediate and Delayed Recall, Wechsler Memory Scale (WMS) Verbal Pair Associate Learning Test Immediate and Delayed Recall, Delis-Kaplan Executive Function System Verbal Fluency Conditions 1 and 2, and the Wechsler Adulte Intelligence Scale Fourth Edition Symbol Search and Coding Subsets and Cognitive Drug Research test battery. Mini Mental Test Scores MMSE and Neurpsychiatric Batter test (NTB) and su-bitems may also be utilized to test cognition.

### EXAMPLES

### Example 1—Preclinical and Clinical Studies which Show Scyllo-Inositol Interacts with and Disaggregate Amyloid Aggregates

Preclinical and clinical studies related to scyllo-inositol have been published and demonstrate the safety and activity of scyllo-inositol. See Clinicaltrials.gov and patent publications cited herein, all of which are incorporated by reference. In addition, non-published analyses have been performed and discoveries have been made with respect to the use of scyllo-inositol in a subset of mild AD patients and/or MCI patients having MMSE scores of between 22-26.

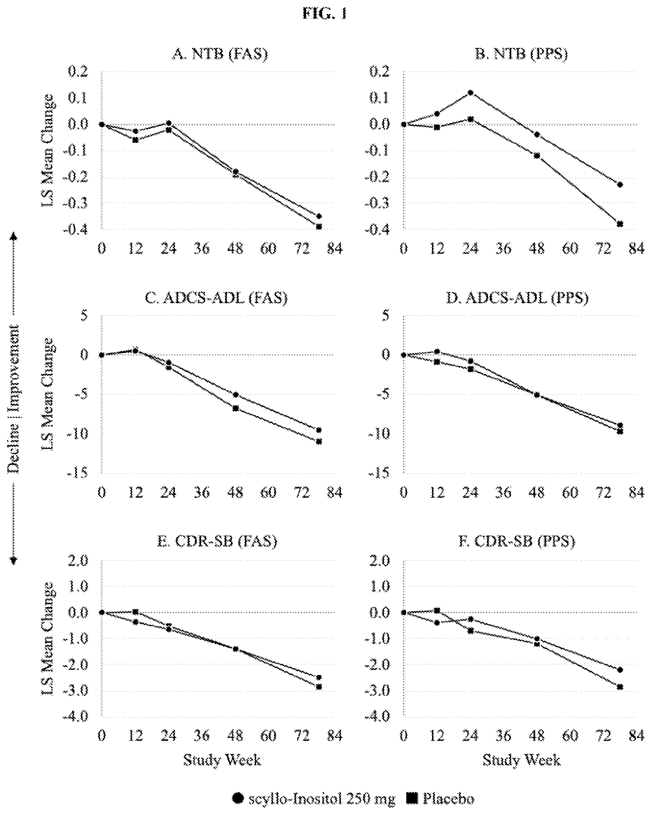

FIGS. 1A-F show the effect of 250 mgs scyllo-inositol Treatment BID in Mild/Moderate AD Patients (MMSE 16-30) in the primary endpoints. NTB. ADCS-ADL, and CDR-SB. The data demonstrates that treatment of Mild and Moderate AD patients with scyllo-inositol for 78 weeks did not improve NTB, ADCS-ADL and CDR-SB scores as a measure of cognition and function. Although a small signal was observed in the NTB scores for the per protocol population.

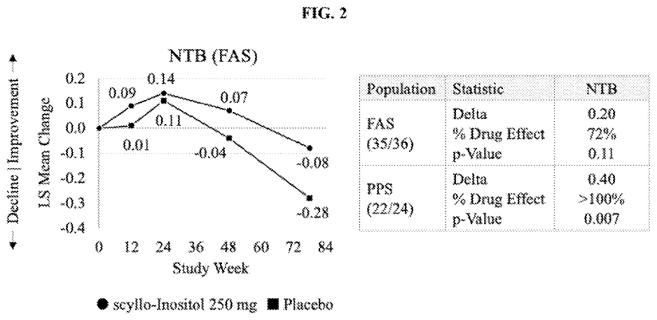

FIG. 2 demonstrates the effect scyllo-inositol treatment of early mild AD patients (MMSE 23-26) for 78 weeks in a pre-specified full and per protocol populations. Following scyllo-inositol treatment, the analysis in the full population showed an improvement in the NTB scores 0f 72% relative to the placebo population. Similarly, the data from the per protocol population showed 100% improvement in the NTB scores relative to placebo. These data show a strong signal that scyllo-inositol treatment improves cognition in early mild AD patients.

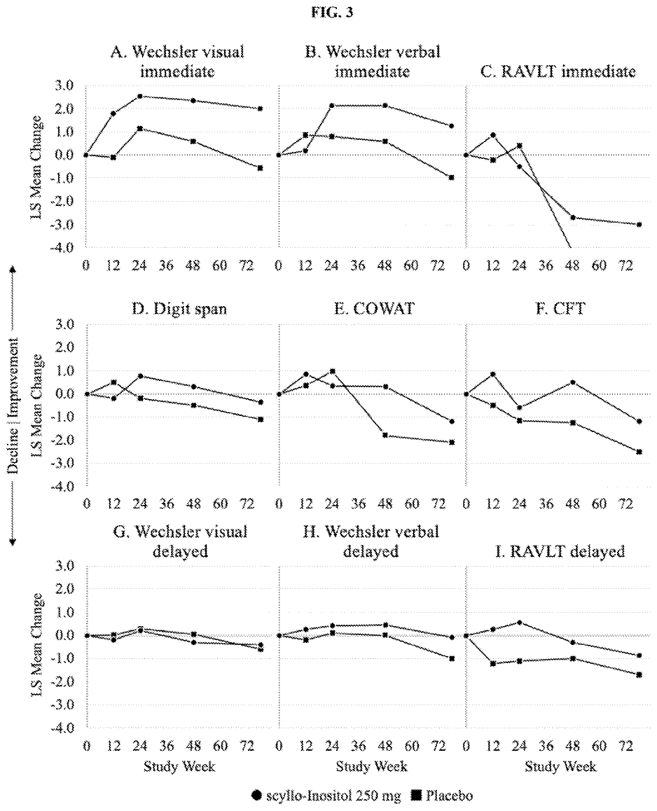

FIGS. 3A-I show the changes in NTB sub-item scores from baseline for the scyllo-inositol and placebo treated Mild AD patient population (MMSE 23-26) for the duration of the study (78 weeks). The data demonstrate that 8 of 9 NTB sub-items improve over 78 weeks with scyllo-inositol treatment. These data show that scyllo-inositol improves a variety of symptoms associated with cognition.

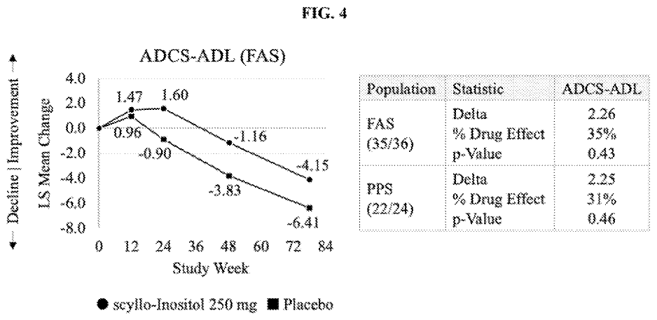

FIG. 4 shows ADCS-ADL change from baseline for scyllo-inositol and placebo treated early-mild AD patients (MMSE 23-26) for 78 weeks. The data shows that scyllo-inositol treatment improved the ADCS-ADL scores throughout the study period of 78 weeks compared to placebo. Scyllo-inositol treatment resulted in a 35% and 31% improvement in ADCS-ADL scores for the full analysis and per protocol populations, respectively. These data suggest that scyllo-inositol improves function in early mild AD patients.

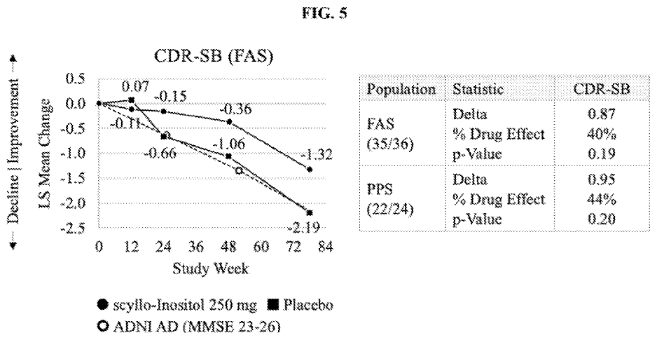

FIG. 5: shows CDR-SB change from baseline for scyllo-inositol and placebo treated early mild AD patients (MMSE 23-26) for 78 weeks. The data shows that scyllo-inositol treatment improved CDR-SB scores for the duration of the 78 week study compared to placebo. Scyllo-inositol treatment improved CDR-SB scores by 40 and 44% relative to placebo for the Full analysis and Per Protocol populations, respectively. These data demonstrate that scyllo-inositol improves cognition and function as measured by CDR-SB test,

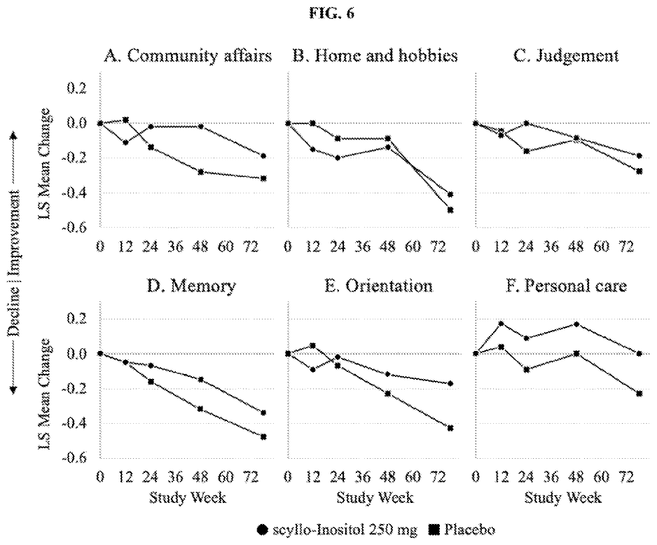

FIGS. 6A-F: show the compares the effect of scyllo-inositol and placebo treatment on the CDR-SB sub-items change from baseline in early mild AD patients in the per protocol population (PPS). These data show that scyllo-inositol improved 5 of the 6 sub-items in the CDR-SB test measuring both cognition and function.

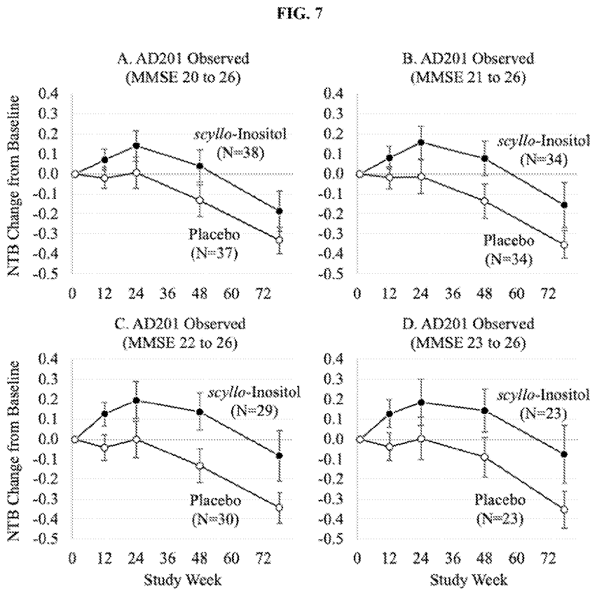

FIGS. 7A-D show the observed changes from baseline in NTB scores with scyllo-inositol treatment of mild AD patients with different MMSE scores ranging from 20 to 26. The data shows that scyllo-inositol was more effect in improvement of NTB scores in patient populations with increasing MMSE scores of up to 23. Ideally, the drug is more effective in patients with MMSE score of 22 or greater.

FIGS. 8A-D show Bootstrap simulated data for the change in NTB scores from baseline with scyllo-inositol treatment of different mild AD patient groups with MMSE scores ranging from 20 to 26. Analysis of the data using the Bootsratp simulation method by increasing N=30 to N=100, showed a similar pattern to the observed data. These data showed that changes in NTB were more apparent the scyllo-inositol treated groups when Bootstrap analysis was performed, further suggesting that by increasing the number of patients would strengthen the analysis.

FIGS. 9A-D show the observed data demonstrating the changes in CDR-SB scores from baseline with scyllo-inositol treatment in different mild AD groups with an MMSE score of 20 to 26. Similarly, scyllo-inositol treatment was more effective in early mild AD patient with MMSE of 22 or greater.

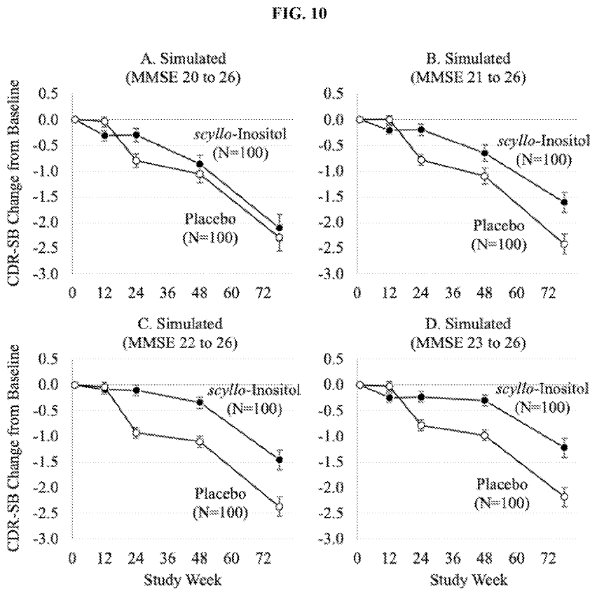

FIGS. 10A-D show bootstrap simulated data demonstrating the changes in CDR-SB scores from baseline with scyllo-inositol treatment in different mild AD groups with an MMSE score range of 20 to 26. The data shows a similar pattern for the treatment effect of scyllo-inositol on CDR-SB test when a Bootstrap simulation analysis was performed. The changes in CDR-SB scores for scyllo-inositol treated group compared to placebo was more apparent when N was increased from 30 to 100 patients in the simulation analysis.

FIGS. 11A-D show a comparison of observed versus simulated data for changes in NTB and CDR-SB scores with scyllo-inositol treatment of mild AD patients with MMSE scores of 22 to 26. FIG. 1A shows AD201 Observed (NTB). FIG. 1B shows simulated bootstrap (NTB). FIG. 1C shows AD201 Observed (CDR-SB). FIG. 1D shows simulated bootstrap (CDR-SB). The Bootstrap simulation used N=100 while the observed set had N=30. Statistical significance was achieved in the treated groups relative to placebo group in the simulated comparison for both endpoints, NTB and CDR-SB. These data demonstrate that scyllo-inositol shows a strong efficacy signal in the improvement of cognition and function in early Mild AD patient with MMSE of 22 or greater.

### Example 2—Clinical Study with a Combination of Scyllo-Inositol and Aducanamab

Clinical studies are conducted in patients having mild cognitive impairment (MCI) and/or mild Alzheimer's disease. Enrolled patients having MCI and/or mild Alzheimer's disease are pretreated with scyllo-inositol for 4 weeks followed by co-administration of scyllo-inositol (250 mgs or 500 mgs BID) in combination with anti-amyloid beta antibody therapies to MCI and mild AD to mitigate the concerns associated with ARIA from monoclonal therapy alone. The clinical study will measure the effectiveness of the drug combination in enhancing the reduction in amyloid beta burden in the brain resulting in improved efficacy and safety relative to the treatment with either drug alone. The MCI and mild AD patients are distributed between 3 cohorts and treated as described below:
a. Cohort #1: Patients will be treated with placebo for 4 weeks and then followed with a combination of placebo and escalating doses of aducanamab alone, beginning with 1 mg/kg (4 weeks), 3 mg/kg (4 weeks), 6 mg/kg (4 weeks) and 10 mg/kg for the remaining period of the study (24 weeks). The patients will be evaluated for ARIA, amyloid beta burden, memory, cognition and function, and safety parameters
b. Cohort #2: Patients will be treated with 250 mg BID of scyllo-inositol alone for 4 weeks, then followed a combination of 250 mg BID of scyllo-inositol and escalating doses of aducanamab beginning with 1 mg/kg (4 weeks), then 3 mg/kg (4 weeks), then 6 mg/kg (4 weeks) and then a final dose of 10 mg/kg for the remaining period of the study (24 weeks). The patients will be evaluated for ARIA, amyloid beta burden, memory, cognition and function, and safety parameters.
c. Cohort #3: Patients will be treated 500 mgs BID of scyllo-inositol alone for 4 weeks, then followed with a combination of 250 mg BID of scyllo-inositol and escalating doses of aducanamb, beginning with 1 mg/kg (4 weeks), 3 mg/kg (4 weeks), 6 mg/kg (4 weeks) and 10 mg/kg for the remaining period of the study (24 weeks). The patients will be evaluated for ARIA, amyloid beta burden, memory, cognition and function, and safety parameters.

The results will demonstrate that combination treatment of scyllo-inositol and aducanamab for a period of 36 weeks will result in:

Decreased ARIA in comparison to treatment with aducanumab alone.

Decreased amyloid beta burden in the brain compared to treatment of aducanumab alone.

Improved memory, cognition and function in comparison to treatment of aducanumab.

Improvement in amyloid beta biomarkers in the CSF such as amyloid beta 42/40 ratio, Tau and phosphorylated Tau.

The specific clinical protocol follows the same protocols used in the ADUHELM clinical studies for measuring each of plaque levels, ARIA impact, Tau protein in CSF and exposure response relationships.

### Example 3—Studies Conducted on the Combination of Scyllo-Inositol and Lecanemab

Clinical studies are conducted in patients having mild cognitive impairment (MCI) with MMSE of 26-30 and/or mild Alzheimer's disease with MMSE of 22-26. Enrolled patients having MCI and/or mild Alzheimer's disease are treated with placebo or with scyllo-inositol in combination with lecanemab.

A subset of patients having mild AD with MMSE scores ranging from either 22-26 or with scores ranging from 23-26 are recruited for the clinical trial. The number of subjects in each arm includes at least 45 to 50 patients. Three arms include (1) said patients are treated with placebo for four weeks followed by the selected immunotherapeutic for thirty six (36) weeks; (2) said patients are treated with 250 mgs BID scyllo-inositol for four weeks followed by thirty six (36) weeks with 250 mgs BID scyllo-inositol and the prescribed dosing amount and doing frequency of the selected immunotherapeutic and (3) said patients are treated with 500 mgs BID of scyllo-inositol for four weeks followed by thirty six (36) weeks of a combination of 250 mgs BID of scyllo-inositol and the prescribed dosing amount and frequency of the selected immunotherapeutic. The primary endpoint measure is the incidence of ARIA E and H at −4, 0, 14, 26 and 36 weeks. The secondary endpoints include the measurement of Aβ burden (PET scan) at −4 and 36 weeks with other endpoints inclusive of NTB, cDR-SB, MMSE. Biomarkers such as tau and P-tau may be measured as well. The incidence or prevalence of subjects having ARIA in the immunotherapeutic treated patients is assumed to be about 22-30% ARIA E and H and will also be based upon/compared to the data obtained for ARIA in the reported clinical studies for lecanemab in both Apo E4 negative and positive subjects.

Clinical studies can also be performed on the combination of decanemab and the other known monoclonal antibodies to treat Alzheimer's disease and scyllo-inositol following protocols similar to those described above for the lecanemab and aducanumab.

1. A method for treating MCI and Alzheimer's disease in a human patient, comprising administering a pharmaceutically effective amount of a recombinant, fully human, anti-amyloid beta monoclonal antibody and a pharmaceutically effective amount of scyllo-inositol.

2. The method according to claim 1, wherein the monoclonal antibody is selected from aducanumab or lecanemab or donanemab.

3-4. (canceled)

5. The method according to claim 1, wherein scyllo-inositol is administered orally at a dosing range of between 125 to 250 mgs BID or 250 mg QD or 500 mg QD.

6. (canceled)

7. A method of treating an Alzheimer's patient having the confirmed presence of amyloid pathology and mild cognitive impairment or mild dementia stage of disease consistent with Stage 3 or Stage 4 Alzheimer's disease comprising the administration of between about 1 mg/kg to about 10 mg/kg of aducanumab as an IV infusion over an hour period every four weeks and at least 21 days apart and comprising the administration of an effective amount of scyllo-inositol.

8. The method according to claim 7, wherein infusion 1 and 2 are 1 mg/kg and infusion 3 and 4 are 3 mg/kg and infusion 5 and 6 are 6 mg/kg and infusion 7 and beyond are 10 mg/kg and wherein infusions are 4 weeks apart and wherein scyllo-inositol is administered BID at a strength of 100 mgs or 125-250 mg BID or 250 mg QD or 500 mg QD.

9. The method according to claim 7, wherein scyllo-inositol enhances the efficacy of aducanumab measured by NTB, CDR-SoB, MMSE, and associated cognition and function sub-items.

10. The method according to claim 7, wherein scyllo-inositol enhances the cognition of a subject being treated with the combination compared to the treatment of the subject on aducanumab alone.

11. The method according to claim 7, wherein scyllo-inositol reduces the dosing requirement of aducanumab so that infusions 7 and beyond are 6 mg/kg.

12. The method according to claim 7, wherein there is a reduction of ARIA aducanumab associated events for the combination treatment versus treatment with aducanumab alone at the same infusion doses.

13-15. (canceled)

16. A method of decreasing amyloid beta burden in the brain of a patient who has mild Alzheimer's disease comprising administering a pharmaceutically effective amount of scyllo-inositol in combination with a pharmaceutical effective amount of a monoclonal antibody to said patient and wherein mild Alzheimer's disease is inclusive of patients having MMSE scores of 22 to 26.

17-19. (canceled)

20. The method according to claim 16, wherein the patient is pretreated with scyllo-inositol before undergoing monoclonal antibody therapy at a dose of 125-250 mgs BID or 250 mg QD or 500 mg QD.

21. The method according to claim 20, wherein the pre-treatment period is about two to six weeks.

22. The method according to claim 21, wherein the duration of the combination treatment is at least six months or for the full duration of antibody treatment.

23-30. (canceled)
